(19) United States
(12) Patent Application Publication
(10) Pub. No.: US 2024/0425434 A1
(43) Pub. Date: Dec. 26, 2024

(54) PREPARATION METHOD FOR 1,3-DISUBSTITUTED ALLENE COMPOUND AT ROOM TEMPERATURE BASED ON METAL CARBENE CATALYTIC SYSTEM

(71) Applicant: SHANGHAI INSTITUTE OF ORGANIC CHEMISTRY, CHINESE ACADEMY OF SCIENCES, Shanghai (CN)

(72) Inventors: Shengming MA, Shanghai (CN); Junzhe XIAO, Shanghai (CN)

(73) Assignee: SHANGHAI INSTITUTE OF ORGANIC CHEMISTRY, CHINESE ACADEMY OF SCIENCES, Shanghai (CN)

(21) Appl. No.: 18/695,741

(22) PCT Filed: Sep. 23, 2022
(86) PCT No.: PCT/CN2022/120980

§ 371 Date: Mar. 26, 2024

(30) Foreign Application Priority Data
Sep. 27, 2021 (CN) 202111135110.5

(51) Int. Cl. C07C 2/86; C07C 1/24; C07C 1/26; C07D 211/40; C07F 7/08

(52) U.S. Cl. CPC C07C 2/867; C07C 1/24; C07C 1/26; C07C 2/865; C07D 211/40; C07F 7/0834

(57) ABSTRACT

Disclosed are a preparation method for a 1,3-disubstituted allene compound at room temperature based on a metal carbene catalyst, comprising: reacting terminal alkynes, aldehydes and amines in an organic solvent under the action of a gold catalyst and a molecular sieve, and then synthesizing a 1,3-disubstituted allene compound at room temperature. The method of the present invention is simple to operate, raw materials and reagents are easily obtained, reaction conditions are mild, substrate universality is wide, functional group compatibility is good, yield is high (36-93%), the method is scalable (11 g), and practicability is strong. The 1,3-disubstituted allene compound obtained in the present invention may be used as an important intermediate to construct δ-caprolactone, trans-allyl alcohol, other allene-derived compounds and natural product molecules and the like.

### TECHNICAL FIELD

The invention belongs to the technical field of chemical synthesis and in particular relates to a method for efficiently synthesizing 1,3-disubstituted allene compound at room temperature.

### BACKGROUND OF THE INVENTION

Allenes are a class of organic compounds containing cumulative diene functional groups. (Ref: (a) D. R. Taylor, Chem. Rev. 1967, 67, 317-359; (b) N. Krause, A. S. K. Hashmi, Modern Allene Chemistry, Wiley-VCH, Weinheim, 2004.) Due to the high reactivity and abundant reaction sites of the accumulated diene functional groups, allenes have high chemical synthesis value. At the same time, allenes are widely distributed in a variety of natural products, pharmaceutically active molecules and materials science, and are a very important class of compounds. Therefore, the efficient synthesis of allenes has always been a hot spot for synthetic chemists. The ATA (Allenation of Terminal Alkynes) reaction is a one-step method for efficiently synthesizing allenes from aldehydes or ketones, terminal alkynes, and amines. (Ref: (a) P. Crabbé, H. Fillion, D. André, J.-L. Luche, J. Chem. Soc., Chem. Commun. 1979, 859-860; (b) J. Kuang, S. Ma, J. Org. Chem. 2009, 74, 1763-1765; (c) J. Kuang, S. Ma, J. Am. Chem. Soc. 2010, 132, 1786-1787; (d) S. Kitagaki, M. Komizu, C. Mukai, Synlett 2011, 8, 1129-1132; (e) J. Kuang, H. Luo, S. Ma, Adv. Synth. Catal. 2012, 354, 933-944; (f) X. Tang, C. Zhu, T. Cao, J. Kuang, W. Lin, S. Ni, J. Zhang, S. Ma, Nat. Commun. 2013, 4, 2450; (g) X. Huang, T. Cao, Y Han, X. Jiang, W. Lin, J. Zhang, S. Ma, Chem. Commun. 2015, 51, 6956-6959. (h) D. M. Lustosa, S. Clemens, M. Rudolph, A. S. K. Hashmi, Adv. Synth. Catal. 2019, 361, 5050-5056; For an account, see: (i) X. Huang, S. Ma, Acc. Chem. Res. 2019, 52, 1301-1312.) 1979, Crabbe research group first reported this method, but since the reaction can only be applied to the synthesis of terminal allenes from paraformaldehyde and the yield is low, it has not received enough attention and development over a long period of time. In 2009, our research group optimized the conditions of Crabbe's, increased the yield of the reaction, and began to gradually broaden the scope of application of the reaction to general aldehydes and ketones. However, there are still two difficulties in the ATA reaction that have not been resolved: 1) the reaction consists of two steps, A³ coupling reaction to generate propargylamine followed by 1,5-hydrogen migration followed by β-elimination to generate allene. The step of 1,5-hydrogen migration needs to overcome a very high energy barrier, and usually needs to be carried out at a high temperature of more than one hundred degrees. Such harsh conditions not only increase energy consumption and safety hazards, but also often result in very low yields or no products for unstable allene synthesis. 2) The in situ generated water and imines in the reaction will reduce the catalytic performance of the metal species, resulting in the vast majority of cases, the metal catalysts used in the ATA reaction are not catalytic.

### SUMMARY OF THE INVENTION

In order to overcome the above-mentioned problems in the prior art, the object of the present invention is to provide a method for synthesizing 1,3-disubstituted allene compound in one step at room temperature, that is, through the reaction of terminal alkynes (2), aldehydes (1) and amines (3) in an organic solvent under the action of a gold carbene catalyst and a molecular sieve, and a 1,3-disubstituted allene compound is synthesized in the next step at room temperature.

The present invention is achieved by adopting the following specific technical solutions:

The invention provides a method for synthesizing 1,3-disubstituted allene compound in one step at room temperature, comprising: terminal alkynes (2) with different substituents, aldehydes (1) and amines (3) in an organic solvent under the action of a gold carbene catalyst a and molecular sieve to generate 1,3-disubstituted allene compound at room temperature, and the reaction process is shown in the following reaction formula (a):

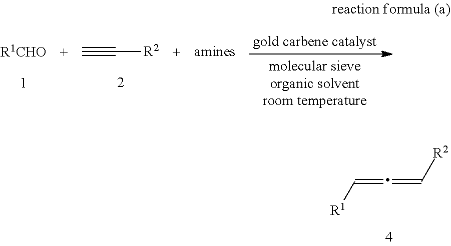

Wherein,
R¹ is alkyl group, alkyl group with functional groups, phenyl, aryl and heterocyclic groups;
R² is alkyl group, alkyl group with functional groups, phenyl, aryl and heterocyclic groups;

The functional groups in R¹ and R² are selected from carbon-carbon double bonds, halogen atoms, hydroxyl, silyl ether, carbonyl, nitrile, ester and amido groups; the said aryl groups are phenyl and polyphenyl cyclosubstituented groups with electron-donating or electron-withdrawing substituents in the ortho, meta and para positions; the said heterocyclic groups are furyl, benzofuryl, thienyl, pyridyl, indolyl and indazolyl.

Preferably,
R¹ is C1-C10 alkyl group, C1-C10 alkyl group with functional groups, phenyl, aryl and heterocyclic groups;
R² is C1-C10 alkyl group, C1-C10 alkyl group with functional groups, phenyl, aryl and heterocyclic groups;

The functional groups in R¹ and R² are selected from carbon-carbon double bonds, halogen atoms, hydroxyl, silyl ether, carbonyl, nitrile, ester and amido groups; the said aryl groups are phenyl and polyphenyl cyclosubstituented groups with electron-donating or electron-withdrawing substituents in the ortho, meta and para positions; the said electron-donating substituents comprises alkyl, methoxy, benzyloxy and boronate groups, and the said electron-withdrawing substituents comprises halogen, nitrile, ester, trifluoromethyl and nitro groups; the said heterocyclic groups are furyl, benzofuryl, thienyl, pyridyl, indolyl and indazolyl.

Further preferably,
R¹ is C1-C10 linear alkyl, C3-C10 cycloalkyl, C1-C10 linear alkyl with functional groups, C3-C10 cycloalkyl with functional groups, phenyl, aryl and heterocyclyl;
R² is C1-C10 linear alkyl, C3-C10 cycloalkyl, C1-C10 linear alkyl with functional groups, C3-C10 cycloalkyl with functional groups, phenyl, aryl and heterocyclyl;

The functional groups in R¹ and R² are selected from carbon-carbon double bonds, halogen atoms, hydroxyl, silyl ether, carbonyl, nitrile, ester and amido groups; the said aryl groups are phenyl and polyphenyl cyclosubstituented groups with electron-donating or electron-withdrawing substituents in the ortho, meta and para positions; the said electron-donating substituents comprises alkyl, methoxy, benzyloxy and boronate groups, and the said electron-withdrawing substituents comprises halogen, nitrile, ester, trifluoromethyl and nitro groups; the said heterocyclic groups are furyl, benzofuryl, thienyl, pyridyl, indolyl and indazolyl.

Further preferably,

R¹ is selected from n-pentyl, isobutyl, cyclohexyl, n-octyl, phenyl, p-methoxyphenyl, p-benzyloxyphenyl, p-diboronic acid pinacol ester phenyl, p-fluorophenyl, p-chlorophenyl, p-bromophenyl, p-iodophenyl, p-nitrilophenyl, p-esterphenyl, p-trifluoromethylphenyl, p-nitrophenyl, naphthyl, phenanthrenyl, pyrenyl, furanyl, benzofuranyl, thienyl, pyridyl, indolyl and indazolyl.

R² is selected from cyclopropyl, n-pentyl, n-hexyl, cyclohexyl, n-octyl, n-decyl, benzyl, buten-4-yl, 4-chlorobutyl, 2-bromoethyl, (dimethyl tert-butylsiloxy) pentyl, 5-hydroxypentyl, 4-nitrilbutyl, 3-methylesterpropyl, tert-butoxycarbonyl protected piperidinyl, (p-methoxybenzoyl)propyl and phenyl.

As a further improvement, the specific operation steps of the present invention are as follows:

Under an argon atmosphere, molecular sieves, gold carbene catalysts and a certain volume of organic solvent are added in sequence to the dry reaction tube. The aldehydes (1), amines (3) and terminal alkynes (2) are then added in sequence with stirring. Reaction is carried out for 24-72 hours at room temperature. After the reaction is complete, the reaction mixture was filtered through a short silica gel column and washed with a certain volume of diethyl ether. After the mixture was concentrated, it was subjected to flash column chromatography to obtain 1,3-disubstituted allene compound.

Wherein, the organic solvent of a certain volume refers to the amount of the terminal alkynes (2) shown in the formula (a) as a benchmark, the amount of the organic solvent is 0.5-5 mL/mmol; preferably, 1 mL/mmol.

Wherein, the room temperature refers to 10-60° C.; preferably, 10-40° C.; more preferably, 25-35° C.

Wherein, the said certain volume of diethyl ether refers to the usage amount of the terminal alkynes (2) shown in the formula (a) as a benchmark, the amount of the said diethyl ether is 10-100 mL/mol; preferably, it is 30 mL/mol.

As a further improvement, the amines (3) described in the present invention is morpholine(3a), piperidine(3b), pyrrolidine(3c), 1,2,3,4-tetrahydroquinoline(3d), 1,2,3,4-tetrahydroisoquinoline(3e), 1-methyl-1,2,3,4-tetrahydroisoquinoline(3f), diethylamine(3g), diallylamine(3h), dicyclohexylamine(3i) and diisopropylamine(3j); preferably, 1-methyl-1,2,3,4-tetrahydroisoquinoline(3f).

As a further improvement, the gold carbene catalyst described in the present invention is selected from one or more of the following structures Au1-Au3. Wherein, R is C1-C30 alkyl group, C1-C30 alkyl group with functional groups, phenyl, aryl, and heterocyclic; the functional group is selected from carbon-carbon double bond, carbon-carbon triple bond, halogen atom, hydroxyl, carboxyl, amino, silyl ether, carbonyl, nitrile, ester and amido groups; the said aryl refers to phenyl and polyphenyl cyclosubstituents with electron-donating or electron-withdrawing substituents in the ortho, meta, and para positions; the electron-donating substituents include alkyl, alkoxy and boronate groups, and the electron-withdrawing substituents include halogen, nitrile, ester, trifluoromethyl and nitro groups.

Among them, X is a counter anion, including halogen anion, hydroxide anion, bis(trifluoromethanesulfonyl)imide anion, methanesulfonate anion, trifluoromethanesulfonate anion, p-methylbenzenesulfonate anion, perchlorate anion, tetrafluoroborate anion, hexafluorophosphate anion and hexafluoroantimonate anion.

Preferably, the gold carbene catalyst is Au3, and the counter anion is bis(trifluoromethanesulfonyl)imide anion.

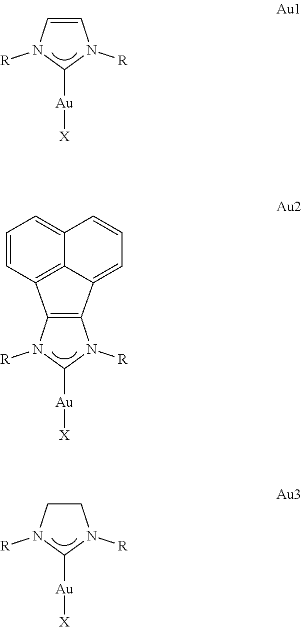

As a further improvement, the gold carbene catalyst of the present invention is selected from one or more of Au3a, Au3b, Au3c, Au3d and Au3e; wherein, the structures of the Au3a, Au3b, Au3c, Au3d and Au3e are as follows:

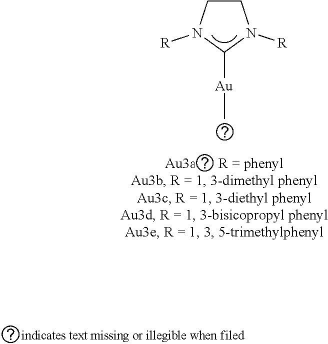

As a further improvement, the molecular sieve described in the present invention is composed of 3 Å molecular sieve, 4 Å molecular sieve and 5 Å molecular sieve; preferably, is 5 Å molecular sieve.

As a further improvement, the organic solvent described in the present invention is selected from one or more of 2,2,2-trifluoroethanol, 2,2,3,3-tetrafluoropropanol, 2,2,3,3,3-pentafluoropropanol and 1,1,1,3,3,3-hexafluoroisopropanol; preferably, 2,2,2-trifluoroethanol.

As a further improvement, the molar ratio of the terminal alkynes (2), aldehydes (1), amines (3) and gold carbene catalyst is 1.0:(1.0-1.8):(1.0-1.4):(0.01-0.1); Preferably, it is 1.0:1.8:1.4:0.05.

As a further improvement, the amount of the molecular sieve used is 100-500 mg/mmol; preferably, 250 mg/mmol, based on the amount of the terminal alkynes (2).

As a further improvement, the amount of the organic solvent used is 0.5-5 mL/mmol; preferably, 1 mL/mmol, based on the amount of the terminal alkynes (2).

Under the gold carbene catalytic conditions of the present invention, the following two technical difficulties in the synthesis of 1,3-disubstituted allene compound are mainly overcome, and the reaction process is shown in the following formula (b):

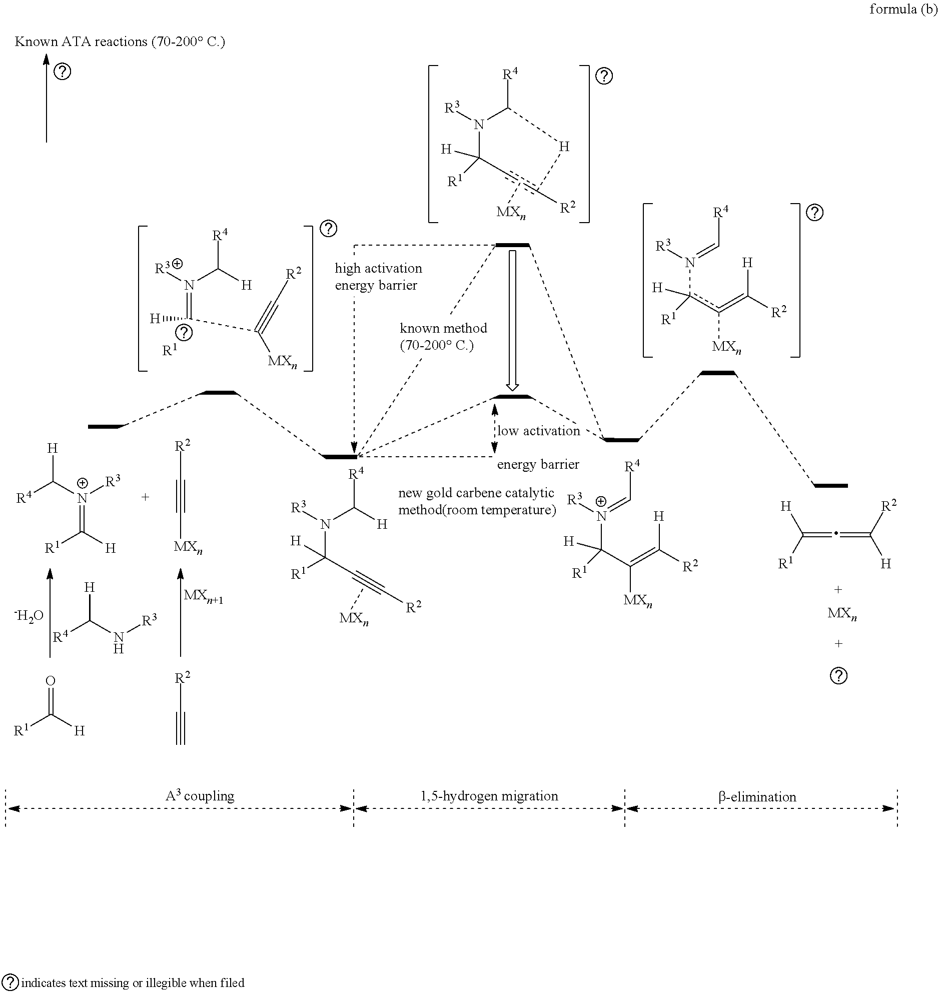

1) In the known method, A³ coupling reaction generates propargyl amine compounds, and the 1,5-hydrogen migration step is the determining step, which needs to overcome a very high activation energy barrier, so the temperature of the reaction is usually at 70-200° C. This not only increases energy loss and security risks, but also often results in very low yields or no products for unstable allene synthesis.

2) The in situ generated water and imines in the reaction will reduce the catalytic performance of the metal species, resulting in the vast majority of cases, the metal catalysts used in the ATA reaction are not catalytic

The present invention can effectively overcome the above technical difficulties by developing a gold carbene catalytic method, and successfully realize the synthesis of 1,3-disubstituted allene compound at room temperature.

The present invention proposes the following possible mechanism for the reaction described in the present invention, as shown in formula (c):
(1) The terminal alkynes (2) reacts with the gold carbene catalyst (Au3d) under the action of a base to generate an alkyne-based gold intermediate I;
(2) In situ dehydration of aldehyde (1) and 1-methyl-1,2,3,4-tetrahydroisoquinoline (3f) to generate imine intermediate II;
(3) The 1,2-addition reaction of the alkyne-based gold intermediate I to the imine intermediate II generates gold carbene coordinated propargylamine intermediates IIIA and IIIB;
(4) Gold carbene coordinated propargylamine intermediates IIIA undergoes 1,5-hydrogen migration at 3-position to obtain the alkenyl gold intermediate IVA; Gold carbene coordinated propargylamine intermediates IIIB undergoes 1,5-hydrogen migration at 1-position to obtain the alkenyl gold intermediate IVB;
(5) Alkenyl gold intermediates IVA and IVB undergo β-elimination reactions to finally generate allenes, and at the same time regenerate the active species of gold carbene catalysts.

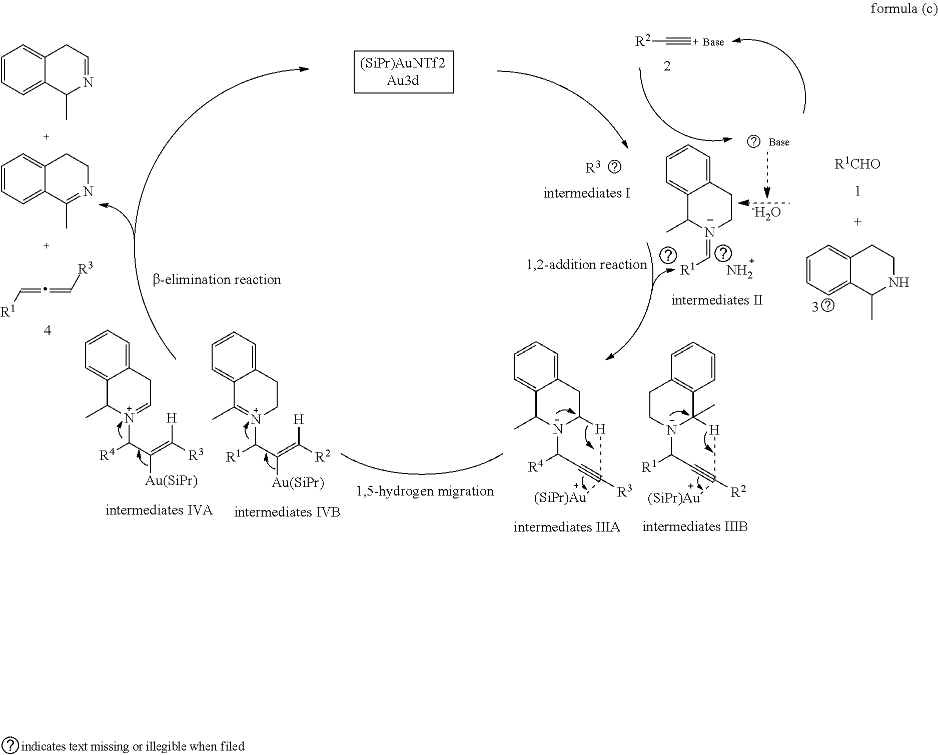

The present invention also provides a class of 1,3-disubstituted allene compound, the structure of which is shown in formula 4

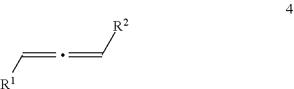

The definitions of R¹ and R² are the same as in the reaction formula (a).

The list of newly prepared compounds in the synthesis process of the present invention is shown in Table 1:

TABLE 1

| 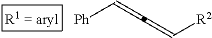 | R² = | n-C [symbol] 84% | n-C₁₀H₃₁ 80% | 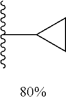 [SMILES: *C1CC1] | | Bn 54% | Ph 62% |
|---|---|---|---|---|---|---|---|
| 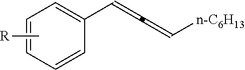 | R = | 4-F 85% | 4-Cl 81% | 4-Br 77% | 4-I 70% | 4-OMe 78% | 4-CN 78% |

| | | | | |
|---|---|---|---|---|
| 4-CO₂Me 77% | 4-CF₃ 80% | 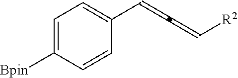 | R² = | 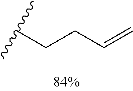 [SMILES: *CCC=C] |

| | |
|---|---|
| 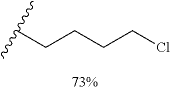 [SMILES: *CCCCCl] | 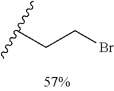 [SMILES: *CCBr] |

| | | |
|---|---|---|
| 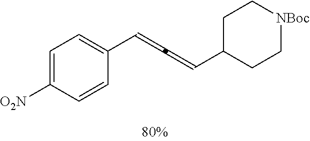 | 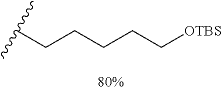 [SMILES: *CCCCCO[Si](C)(C)C(C)(C)C] | 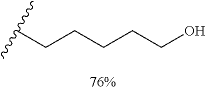 [SMILES: *CCCCCO] |

| | |
|---|---|
| 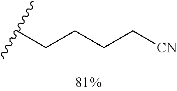 [SMILES: *CCCCC#N] | 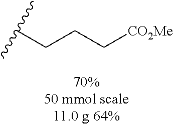 [SMILES: *CCCC(=O)OC] |

| | | |
|---|---|---|
| 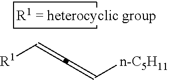 | 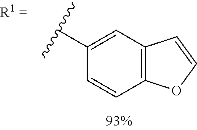 [SMILES: *c1ccc2occc2c1] | 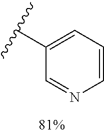 [SMILES: *c1cccnc1] |

| | | |
|---|---|---|
| 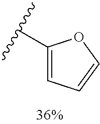 [SMILES: *c1ccco1] | 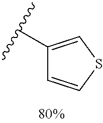 [SMILES: *c1ccsc1] | 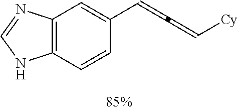 |

| | |
|---|---|
| 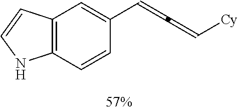 | 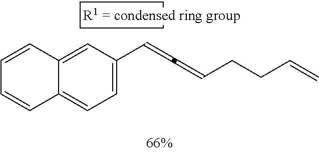 |

| | |
|---|---|
| 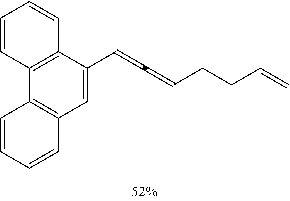 | 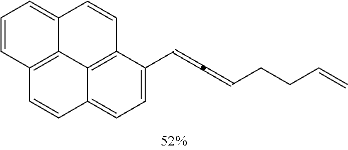 |

| | | | | | | | |
|---|---|---|---|---|---|---|---|
| 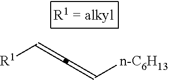 | R¹ = | n-C [symbol] H₁₇ 83% | n- C [symbol] H₁₁ 65% | i-Bu 82% | Cy 78% | 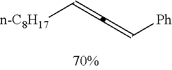 | 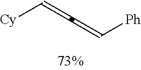 |

| | | | | |
|---|---|---|---|---|
| 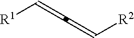 | R¹ = 4-ClC [symbol] H₄, R² = n-C₈H₁₇ R¹ = 4-BrC [symbol] H₄, R² = n-C₈H₁₇ | 76% 75% | 52% 53% | 0. [symbol] equivalent ZuI₂ methylbenzene, 103° C., Ar |
| | R¹ = 4-F [symbol] CC [symbol] H₄, R² = n-C₁₀H₂₁ | 78% | 58% | |
| | R¹ = 3-Thienyl, R² = n-C [symbol] H₁₇ | 86% | 30% | |

[symbol]

indicates data missing or illegible when filed

The present invention also provides the application of 1,3-disubstituted allene compound (4) in the preparation of δ-caprolactone, trans-allyl alcohol, other allene-derived compounds, and natural product molecules, as shown in Table 2:

TABLE 2

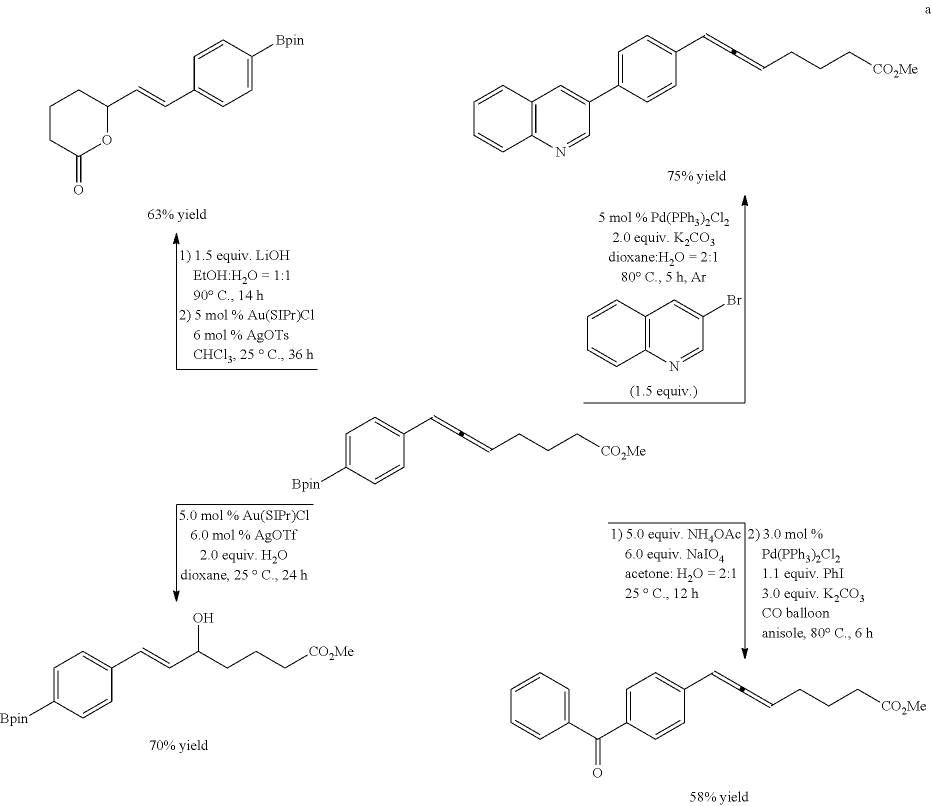

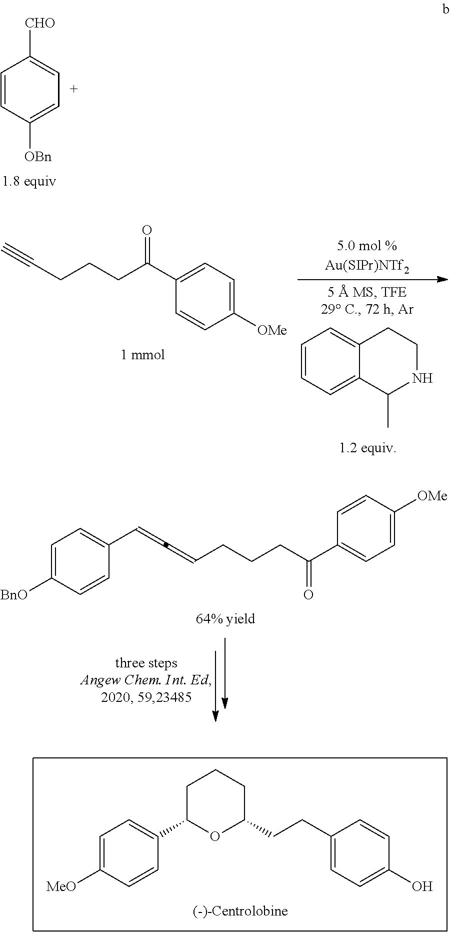

The comparison list of the method described in the present invention and original method:

TABLE 3

| | | Technical solution of the present |
|---|---|---|
| Type | Prior art: | invention: |
| 1. Different types of | ZnI₂ | (SIPr)AuNTf₂ |
| catalysts: | | |
| 2. Different amount | Use an equivalent catalyst | Use catalytic amounts of catalysts |
| of catalysts: | | |
| 3. Different amines | 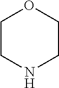 | 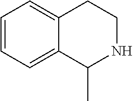 |
| 4. Different organic | methylbenzene | 2,2,2-trifluoroethanol |
| solvent: | | |
| 5. Different reaction | 130-150° C. | 25-60° C. |
| temperature: | | |
| 6. Different energy | There are high barriers to overcome for that | Experiencing a relatively low energy |
| barrier of the | to happen | barrier can occur smoothly |
| rate-determining | | |
| step of 1,5-hydrogen | | |
| migration: | | |
| 7. Different total | A moderate yield is obtained | Get medium to excellent yield |
| reaction yield: | | |
| Example: | 52% | 76% |
| 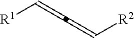 | | |
| R¹ = 4-ClC₈H₄, | | |
| R² = n-C₈H₁₇ | | |
| R¹ = 4-BrC [symbol] H₄, | 53% | 75% |
| R² = n-C₈H₁₇ | | |
| R¹ = 4-F₃CC₈H₄, | 58% | 78% |
| R² = n-C₁₀H₂₁ | | |
| R¹ = 3-Thienyl, | 30% | 86% |
| R² = n-C [symbol] H₁₇ | | |

[symbol]

indicates data missing or illegible when filed

The innovations of the present invention include:

(1) The reaction of the present invention starts from the simple and easy-to-obtain terminal alkynes(2), aldehydes(1) and amines(3), based on the gold carbene catalytic system, and undergoes A³ coupling reaction, 1,5-hydrogen migration reaction and β-elimination reaction, successfully realized the synthesis of 1,3-disubstituted allene at room temperature.

(2) The present invention uses gold carbene (Au3d) as a metal catalyst, 1-methyl-1,2,3,4-tetrahydroisoquinoline (3f) as an amine, and 2,2,2-trifluoroethanol as an organic Solvent, which successfully overcomes or breaks through the technical barriers and limitations of the original ATA reaction at high temperature, and reduces the activation energy barrier of the rate-determining step of 1,5-hydrogen migration in the ATA reaction, making the reaction proceed smoothly at room temperature.

(3) Because of the better catalytic activity of the gold carbene catalyst, under the conditions of gold carbene catalysis, this method can effectively overcome the reduction of the catalytic performance of the metal catalyst by the water and imine generated in situ during the reaction, and achieve the synthesis of 1,3-disubstituted allenes catalyzed by gold carbene at room temperature.

The beneficial effects of the present invention include: the present invention realizes for the first time by using simple and easy-to-obtain terminal alkynes (2), aldehydes (1) and amines (3) as starting materials, under the action of gold carbene catalysts, molecular sieves and organic solvents catalytic synthesis of 1,3-disubstituted allene compound at room temperature. The method of the invention reduces the energy loss and potential safety hazards in the synthesis of allenes, and is suitable for synthesizing unstable allenes. The method of the invention has the advantages of simple operation, easily available raw materials and reagents, mild reaction conditions, wide substrate universality, good functional group compatibility and strong practicability. The 1,3-disubstituted allene compound obtained in the present invention can be used as important intermediates to construct δ-caprolactone, trans-allyl alcohol, other allene-derived compounds, and natural product molecules.

### PREFERRED EMBODIMENTS OF THE INVENTION

The following examples are given to further illustrating the specific solutions of the present invention. The process, conditions, experimental methods, and so on for implementing the present invention are all general knowledge and common knowledge in the field except for the contents specifically mentioned below, and the present invention has no special limitation.

### Example 1

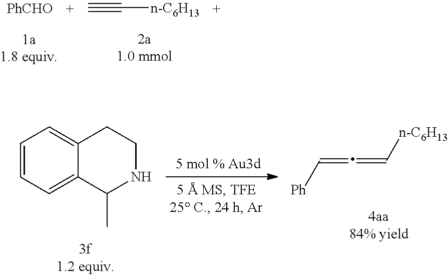

Wherein “mol” represents mole, “equiv.” represents equivalent, “5 Å MS” represents 5 Å molecular sieve, “TFE” represents 2,2,2-trifluoroethanol, “Ar” represents argon atmosphere, “yield” represents yield.

Under the protection of argon atmosphere, 5 Å molecular sieves (250.4 mg), Au3d (43.5 mg, 0.05 mmol) and 2,2,2-trifluoroethanol (1 mL) were added in sequence to a dry reaction tube. Then, under stirring, aldehyde 1a (191.3 mg, 1.8 mmol), 1-methyl-1,2,3,4-tetrahydroisoquinoline 3f (176.6 mg, 1.2 mmol) and terminal alkyne 2a (110.0 mg, 1.0 mmol) were added in sequence. After reacting at room temperature for 24 hours, the reaction solution was filtered through a short silica gel column and the filter cake was washed with diethyl ether (30 mL), concentrated, purified by flash column chromatography (eluent:petroleum ether) to afford an allene product 4aa (168.7 mg, 84% yield): oily liquid; ¹H NMR (400 MHz, CDCl₃): δ=7.36-7.26 (m, 4H, Ar—H), 7.22-7.14 (m, 1H, Ar—H), 6.12 (dt, J₁=6.1 Hz, J₂=3.0 Hz, 1H, ═CH), 5.56 (q, J=6.7 Hz, 1H, ═CH), 2.12 (qd, J₁=7.1 Hz, J₂=2.9 Hz, 2H, CH₂), 1.53-1.41 (m, 2H, CH₂), 1.41-1.18 (m, 6H, CH₂×3), 0.88 (t, J=6.8 Hz, 3H, CH₃); ¹³C NMR (100 MHz, CDCl₃): δ=205.1, 135.1, 128.5, 126.55, 126.52, 95.1, 94.5, 31.6, 29.1, 28.9, 28.7, 22.7, 14.1; IR (neat): ν=2956, 2924, 2854, 1949, 1598, 1495, 1458 cm⁻¹; MS (FI) m/z (%): 200 (M⁺); HRMS Calcd. for C₁₅H₂₀ (M⁺): 200.1560, found 200.1564.

### Example 2

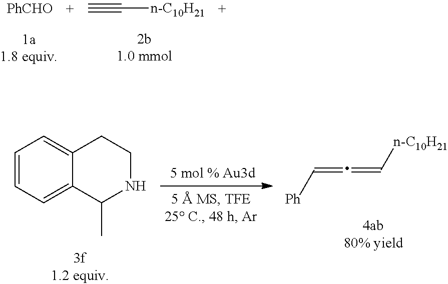

The operation is the same as Example 1. 5 Å molecular sieves (250.1 mg), Au3d (43.4 mg, 0.05 mmol), aldehyde 1a (191.4 mg, 1.8 mmol), 1-methyl-1,2,3,4-tetrahydroisoquinoline 3f (176.5 mg, 1.2 mmol) and terminal alkyne 2b (166.1 mg, 1.0 mmol) were reacted in 2,2,2-trifluoroethanol (1 mL) to afford an allene product 4ab (205.5 mg, 80% yield) (purified by flash column chromatography (eluent:petroleum ether)): oily liquid; ¹H NMR (400 MHz, CDCl₃): δ=7.33-7.25 (m, 4H, Ar—H), 7.21-7.12 (m, 1H, Ar—H), 6.11 (dt, J₁=6.1 Hz, J₂=3.1 Hz, 1H, ═CH), 5.56 (q, J=6.5 Hz, 1H, ═CH), 2.12 (qd, J₁=7.0 Hz, J₂=2.7 Hz, 2H, CH₂), 1.52-1.41 (m, 2H, CH₂), 1.40-1.12 (m, 14H, CH₂×7), 0.88 (t, J=6.8 Hz, 3H, CH₃); ¹³C NMR (100 MHz, CDCl₃): δ=205.1, 135.2, 128.5, 126.56, 126.55, 95.1, 94.5, 31.9, 29.64, 29.60, 29.4, 29.3, 29.20, 29.17, 28.8, 22.7, 14.1; IR (neat): ν=2922, 2852, 1949, 1598, 1495, 1460, 1071 cm⁻¹; MS (70 eV, EI) m/z (%): 256 (M⁺, 1.02), 130 (100).

### Example 3

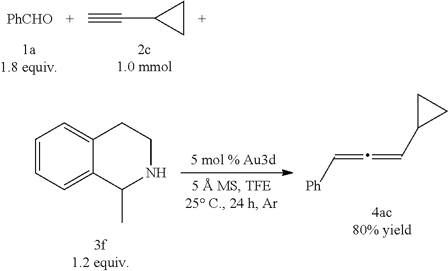

The operation is the same as Example 1. 5 Å molecular sieves (251.0 mg), Au3d (43.6 mg, 0.05 mmol), aldehyde 1a (191.2 mg, 1.8 mmol), 1-methyl-1,2,3,4-tetrahydroisoquinoline 3f (176.8 mg, 1.2 mmol) and terminal alkyne 2c (67.5 mg, 1.0 mmol) were reacted in 2,2,2-trifluoroethanol (1 mL) to afford an allene product 4ac (124.8 mg, 80% yield) (purified by flash column chromatography (eluent:petroleum ether)): oily liquid; ¹H NMR (400 MHz, CDCl₃): δ=7.33-7.23 (m, 4H, Ar—H), 7.21-7.13 (m, 1H, Ar—H), 6.20 (d, J=6.0 Hz, 1H, ═CH), 5.43 (t, J=6.8 Hz, 1H, ═CH), 1.39-1.25 (m, 1H, CH), 0.80-0.68 (m, 2H, CH₂), 0.51-0.37 (m, 2H, CH₂); ¹³C NMR (100 MHz, CDCl₃): δ=204.8, 134.9, 128.5, 126.8, 126.6, 99.4, 96.2, 9.4, 7.0, 6.8; IR (neat): ν=3081, 3004, 1946, 1597, 1492, 1458, 1423, 1251, 1047, 1020 cm⁻¹; MS (70 eV, EI) m/z (%): 156 (M⁺, 86.83), 115 (100).

### Example 4

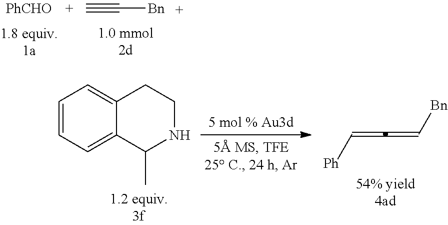

The operation is the same as Example 1. 5 Å molecular sieves (250.9 mg), Au3d (43.4 mg, 0.05 mmol), aldehyde 1a (191.2 mg, 1.8 mmol), 1-methyl-1,2,3,4-tetrahydroisoquinoline 3f (176.5 mg, 1.2 mmol) and terminal alkyne 2d (118.8 mg, 1.0 mmol) were reacted in 2,2,2-trifluoroethanol (1 mL) to afford an allene product 4ad (111.0 mg, 54% yield) (purified by flash column chromatography (eluent:petroleum ether)): oily liquid; ¹H NMR (400 MHz, CDCl₃): δ=7.35-7.24 (m, 8H, Ar—H), 7.24-7.15 (m, 2H, Ar—H), 6.17 (dt, J₁=5.5 Hz, J₂=2.8 Hz, 1H, ═CH), 5.72 (q, J=7.1 Hz, 1H, ═CH), 3.47 (dd, J₁=7.4 Hz, J₂=2.2 Hz, 2H, CH₂); ¹³C NMR (100 MHz, CDCl₃): δ=205.7, 140.0, 134.6, 128.5, 128.4, 126.8, 126.7, 126.3, 94.9, 94.4, 35.5; IR (neat): ν=3028, 1948, 1599, 1493, 1454, 1072, 1028 cm⁻¹; MS (70 eV, EI) m/z (%): 206 (M⁺, 100).

### Example 5

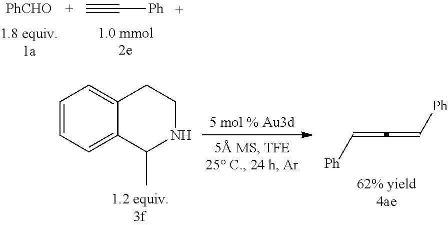

The operation is the same as Example 1. 5 Å molecular sieves (250.1 mg), Au3d (43.3 mg, 0.05 mmol), aldehyde 1a (191.2 mg, 1.8 mmol), 1-methyl-1,2,3,4-tetrahydroisoquinoline 3f (176.6 mg, 1.2 mmol) and the terminal alkyne 2e (102.3 mg, 1.0 mmol) were reacted in 2,2,2-trifluoroethanol (1 mL) to afford an allene product 4ae (118.8 mg, 62% yield) (purified by flash column chromatography (eluent:petroleum ether)): oily liquid; ¹H NMR (400 MHz, CDCl₃): δ=7.43-7.26 (m, 8H, Ar—H), 7.26-7.16 (m, 2H, Ar—H), 6.59 (s, 2H, ═CH×2); ¹³C NMR (100 MHz, CDCl₃): δ=207.7, 133.5, 128.7, 127.3, 127.0, 98.4; IR (neat): ν=3027, 1935, 1596, 1491, 1449, 1254, 1071, 1027 cm⁻¹; MS (70 eV, EI) m/z (%): 192 (M⁺, 100).

### Example 6

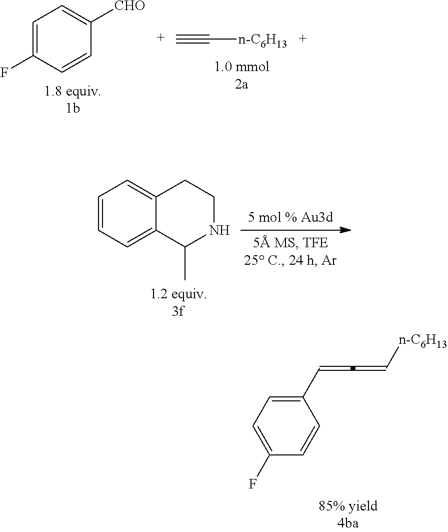

The operation is the same as Example 1. 5 Å molecular sieves (250.6 mg), Au3d (43.2 mg, 0.05 mmol), aldehyde 1b (223.1 mg, 1.8 mmol), 1-methyl-1,2,3,4-tetrahydroisoquinoline 3f (176.9 mg, 1.2 mmol) and terminal alkyne 2a (110.0 mg, 1.0 mmol) were reacted in 2,2,2-trifluoroethanol (1 mL) to afford an allene product 4ba (185.6 mg, 85% yield) (purified by flash column chromatography (eluent:petroleum ether)): oily liquid; ¹H NMR (400 MHz, CDCl₃): δ=7.29-7.17 (m, 2H, Ar—H), 7.03-6.91 (m, 2H, Ar—H), 6.08 (dt, J₁=6.1 Hz, J₂=3.1 Hz, 1H, ═CH), 5.56 (q, J=6.7 Hz, 1H, ═CH), 2.17 (qd, J₁=7.1 Hz, J₂=3.0 Hz, 2H, CH₂), 1.53-1.41 (m, 2H, CH₂), 1.41-1.18 (m, 6H, CH₂×3), 0.88 (t, J=6.6 Hz, 3H, CH₃); ¹³C NMR (100 MHz, CDCl₃): δ=204.9 (d, J=2.5 Hz), 161.7 (d, J=244.5 Hz), 131.1 (d, J=3.2 Hz), 127.9 (d, J=8.1 Hz), 115.4 (d, J=21.4 Hz), 95.3, 93.6, 31.6, 29.1, 28.9, 28.8, 22.7, 14.0; ¹⁹F NMR (376 MHz, CDCl₃): δ=−116.5; IR (neat): ν=2925, 2855, 1948, 1602, 1506, 1463, 1226, 1154 cm⁻¹; MS (70 eV, EI) m/z (%): 218 (M⁺, 2.01), 148 (100).

### Example 7

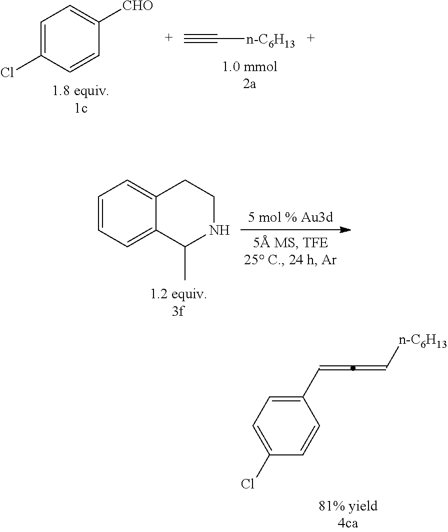

The operation is the same as Example 1. 5 Å molecular sieves (250.6 mg), Au3d (43.4 mg, 0.05 mmol), aldehyde 1c (253.3 mg, 1.8 mmol), 1-methyl-1,2,3,4-tetrahydroisoquinoline 3f (176.5 mg, 1.2 mmol) and terminal alkyne 2a (109.6 mg, 1.0 mmol) were reacted in 2,2,2-trifluoroethanol (1 mL) to afford an allene product 4ca (190.2 mg, 81% yield) (purified by flash column chromatography (eluent:petroleum ether)): oily liquid; ¹H NMR (400 MHz, CDCl₃): δ=7.25 (d, J=8.8 Hz, 2H, Ar—H), 7.20 (d, J=8.4 Hz, 2H, Ar—H), 6.07 (dt, J₁=6.4 Hz, J₂=3.1 Hz, 1H, ═CH), 5.57 (q, J=6.7 Hz, 1H, ═CH), 2.12 (qd, J₁=7.1 Hz, J₂=3.0 Hz, 2H, CH₂), 1.53-1.41 (m, 2H, CH₂), 1.41-1.18 (m, 6H, CH₂×3), 0.88 (t, J=6.8 Hz, 3H, CH₃); ¹³C NMR (100 MHz, CDCl₃): δ=205.2, 133.7, 132.1, 128.6, 127.7, 95.4, 93.7, 31.6, 29.0, 28.8, 28.6, 22.6, 14.0; IR (neat): ν=2924, 2854, 1949, 1490, 1462, 1090, 1012 cm⁻¹; MS (70 eV, EI) m/z (%): 234 (M⁺(³⁵Cl), 1.40), 236 (M⁺(³⁷Cl), 0.62), 129 (100).

### Example 8

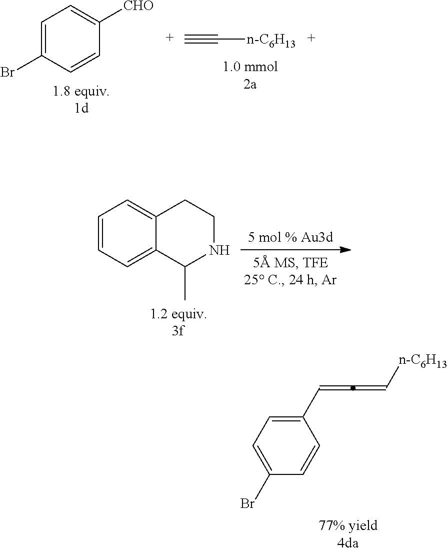

The operation is the same as Example 1. 5 Å molecular sieves (250.2 mg), Au3d (43.3 mg, 0.05 mmol), aldehyde 1d (333.1 mg, 1.8 mmol), 1-methyl-1,2,3,4-tetrahydroisoquinoline 3f (176.8 mg, 1.2 mmol) and terminal alkyne 2a (110.0 mg, 1.0 mmol) were reacted in 2,2,2-trifluoroethanol (1 mL) to afford an allene product 4da (215.8 mg, 77% yield) (purified by flash column chromatography (eluent:petroleum ether)): oily liquid; ¹H NMR (400 MHz, CDCl₃): δ=7.40 (d, J=8.4 Hz, 2H, Ar—H), 7.14 (d, J=8.0 Hz, 2H, Ar—H), 6.11-6.01 (m, 1H, ═CH), 5.56 (q, J=6.7 Hz, 1H, ═CH), 2.12 (qd, J₁=7.1 Hz, J₁=2.4 Hz, 2H, CH₂), 1.52-1.41 (m, 2H, CH₂), 1.41-1.18 (m, 6H, CH₂×3), 0.88 (t, J=6.8 Hz, 3H, CH₃); ¹³C NMR (100 MHz, CDCl₃): δ=205.2, 134.2, 131.5, 128.0, 120.1, 95.5, 93.8, 31.6, 29.0, 28.8, 28.6, 22.6, 14.0; IR (neat): ν=2924, 2853, 1948, 1486, 1463, 1382, 1069, 1009 cm⁻¹; MS (70 eV, EI) m/z (%): 278 (M⁺(⁷⁹Br), 1.71), 280 (M⁺(⁸¹Br), 1.61), 129 (100).

### Example 9

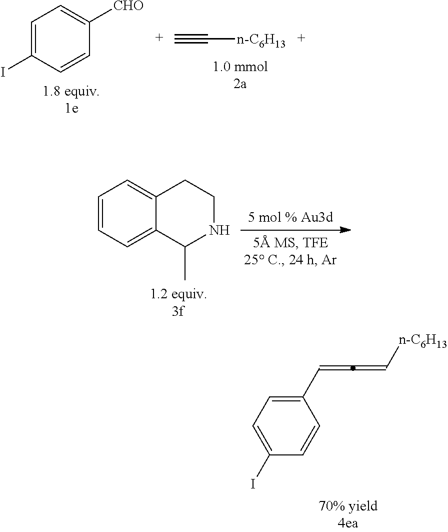

The operation is the same as Example 1. 5 Å molecular sieves (250.4 mg), Au3d (43.5 mg, 0.05 mmol), aldehyde 1e (417.2 mg, 1.8 mmol), 1-methyl-1,2,3,4-tetrahydroisoquinoline 3f (176.5 mg, 1.2 mmol) and terminal alkyne 2a (110.1 mg, 1.0 mmol) were reacted in 2,2,2-trifluoroethanol (1 mL) to afford an allene product 4ea (228.3 mg, 70% yield) (purified by flash column chromatography (eluent:petroleum ether)): oily liquid; ¹H NMR (400 MHz, CDCl₃): δ=7.60 (d, J=8.8 Hz, 2H, Ar—H), 7.02 (d, J=8.0 Hz, 2H, Ar—H), 6.04 (dt, J₁=6.1 Hz, J₂=3.0 Hz, 1H, ═CH), 5.56 (q, J=6.8 Hz, 1H, ═CH), 2.12 (qd, J₁=7.1 Hz, J₂=2.8 Hz, 2H, CH₂), 1.52-1.40 (m, 2H, CH₂), 1.40-1.18 (m, 6H, CH₂×3), 0.88 (t, J=6.6 Hz, 3H, CH₃); ¹³C NMR (100 MHz, CDCl₃): δ=205.3, 137.5, 134.8, 128.3, 95.5, 93.9, 91.4, 31.6, 29.0, 28.8, 28.6, 22.6, 14.1; IR (neat): ν=2923, 2852, 1948, 1483, 1462, 1381, 1058, 1004 cm⁻¹; MS (70 eV, EI) m/z (%): 326 (M⁺, 2.26), 129 (100); HRMS Calcd. for C₁₅H₁₉I (M⁺): 326.0526, found 326.0529.

### Example 10

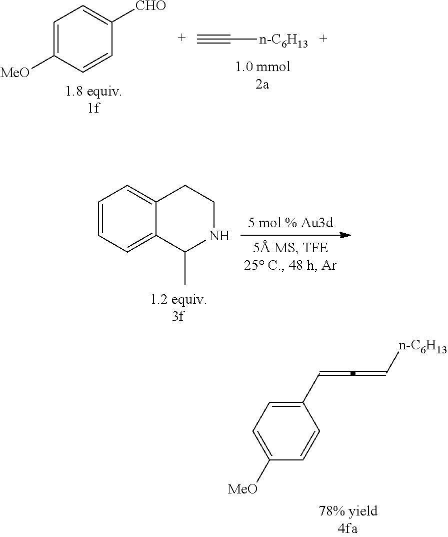

The operation is the same as Example 1. 5 Å molecular sieves (250.4 mg), Au3d (43.3 mg, 0.05 mmol), aldehyde 1f (245.7 mg, 1.8 mmol), 1-methyl-1,2,3,4-tetrahydroisoquinoline 3f (176.6 mg, 1.2 mmol) and terminal alkyne 2a (110.4 mg, 1.0 mmol) were reacted in 2,2,2-trifluoroethanol (1 mL) to afford an allene product 4fa (180.1 mg, 78% yield) (purified by flash column chromatography (eluent:petroleum ether)): oily liquid; ¹H NMR (400 MHz, CDCl₃): δ=7.21 (d, J=8.4 Hz, 2H, Ar—H), 6.84 (d, J=8.4 Hz, 2H, Ar—H), 6.08 (dt, J₁=6.1 Hz, J₂=3.0 Hz, 1H, ═CH), 5.53 (q, J=6.7 Hz, 1H, ═CH), 3.80 (s, 3H, OCH₃), 2.11 (qd, J₁=7.1 Hz, J₂=3.1 Hz, 2H, CH₂), 1.52-1.41 (m, 2H, CH₂), 1.41-1.18 (m, 6H, CH₂×3), 0.88 (t, J=6.8 Hz, 3H, CH₃); ¹³C NMR (100 MHz, CDCl₃): δ=204.4, 158.5, 127.5, 127.4, 114.0, 95.0, 93.9, 55.2, 31.6, 29.1, 28.9, 28.8, 22.6, 14.0; IR (neat): ν=2925, 2854, 1947, 1607, 1510, 1462, 1300, 1243, 1170, 1035 cm⁻¹; MS (70 eV, EI) m/z (%): 230 (M⁺, 20.68), 160 (100).

### Example 11

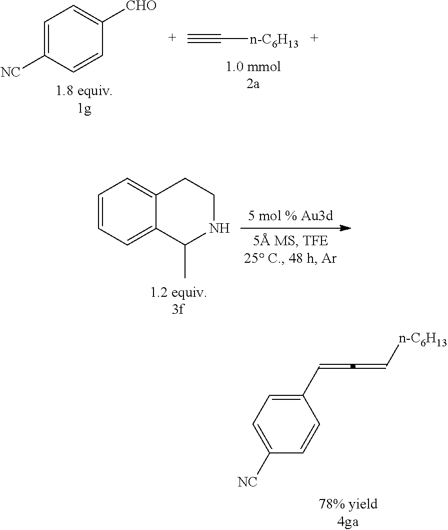

The operation is the same as Example 1. 5 Å molecular sieves (250.3 mg), Au3d (43.3 mg, 0.05 mmol), aldehyde 1g (237.0 mg, 1.8 mmol), 1-methyl-1,2,3,4-tetrahydroisoquinoline 3f (176.3 mg, 1.2 mmol) and terminal alkyne 2a (112.6 mg, 1.0 mmol) were reacted in 2,2,2-trifluoroethanol (1 mL) to afford an allene product 4ga (175.2 mg, 78% yield) (purified by flash column chromatography (eluent:petroleum ether)): oily liquid; ¹H NMR (400 MHz, CDCl₃): δ=7.59-7.53 (m, 2H, Ar—H), 7.38-7.33 (m, 2H, Ar—H), 6.13 (dt, J₁=6.3 Hz, J₂=3.1 Hz, 1H, ═CH), 5.66 (q, J=6.7 Hz, 1H, ═CH), 2.15 (qd, J₁=7.1 Hz, J₂=3.0 Hz, 2H, CH₂), 1.53-1.41 (m, 2H, CH₂), 1.41-1.20 (m, 6H, CH₂×3), 0.88 (t, J=6.8 Hz, 3H, CH₃); ¹³C NMR (100 MHz, CDCl₃): δ=206.5, 140.4, 132.2, 126.9, 119.1, 109.6, 96.0, 93.9, 31.5, 28.9, 28.8, 28.3, 22.5, 14.0; IR (neat): ν=2925, 2854, 2225, 1946, 1604, 1503, 1462, 1394, 1173 cm⁻¹; MS (70 eV, EI) m/z (%): 225 (M⁺, 3.53), 129 (100); HRMS Calcd. for C₁₆H₁₉N (M⁺): 225.1512, found 225.1515.

### Example 12

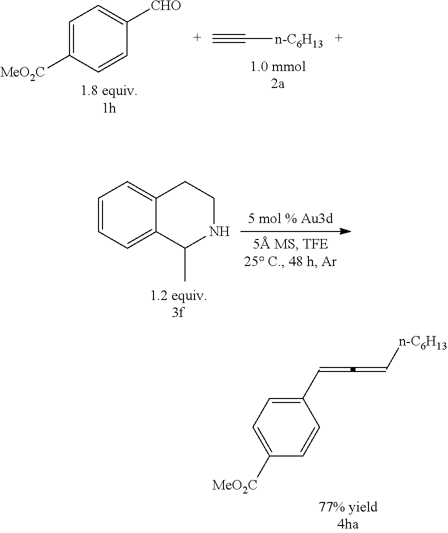

The operation is the same as Example 1. 5 Å molecular sieves (250.8 mg), Au3d (43.3 mg, 0.05 mmol), aldehyde 1h (301.8 mg, 1.8 mmol), 1-methyl-1,2,3,4-tetrahydroisoquinoline 3f (176.8 mg, 1.2 mmol) and terminal alkyne 2a (112.3 mg, 1.0 mmol) were reacted in 2,2,2-trifluoroethanol (1 mL) to afford an allene product 4ha (199.1 mg, 77% yield) (purified by flash column chromatography (eluent:petroleum ether/ethyl acetate=100/1)): oily liquid; ¹H NMR (400 MHz, CDCl₃): δ=7.96 (d, J 8.0 Hz, 2H, Ar—H), 7.33 (d, J 8.0 Hz, 2H, Ar—H), 6.18-6.11 (m, 1H, ═CH), 5.61 (q, J=6.7 Hz, 1H, ═CH), 3.90 (s, 3H, OCH₃), 2.14 (qd, J₁=7.1 Hz, J₂=2.7 Hz, 2H, CH₂), 1.53-1.41 (m, 2H, CH₂), 1.41-1.20 (m, 6H, CH₂×3), 0.88 (t, J=6.6 Hz, 3H, CH₃); ¹³C NMR (100 MHz, CDCl₃): δ=206.3, 166.9, 140.3, 129.8, 128.1, 126.3, 95.5, 94.2, 51.9, 31.6, 29.0, 28.8, 28.5, 22.6, 14.0; IR (neat): ν=2925, 2854, 1947, 1718, 1606, 1434, 1272, 1173, 1105 cm⁻¹; MS (70 eV, EI) m/z (%): 258 (M⁺, 1.27), 129 (100); HRMS Calcd. for C₁₇H₂₂O₂(M⁺): 258.1614, found 258.1613.

### Example 13

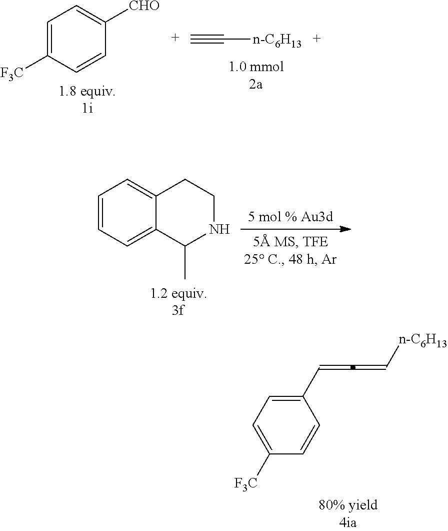

The operation is the same as Example 1. 5 Å molecular sieves (250.5 mg), Au3d (43.4 mg, 0.05 mmol), aldehyde 1i (313.2 mg, 1.8 mmol), 1-methyl-1,2,3,4-tetrahydroisoquinoline 3f (176.8 mg, 1.2 mmol) and terminal alkyne 2a (110.5 mg, 1.0 mmol) were reacted in 2,2,2-trifluoroethanol (1 mL) to afford an allene product 4ia (215.2 mg, 80% yield) (purified by flash column chromatography (eluent:petroleum ether)): oily liquid; ¹H NMR (400 MHz, CDCl₃): δ=7.50 (d, J=8.4 Hz, 2H, Ar—H), 7.34 (d, J=8.0 Hz, 2H, Ar—H), 6.12 (dt, J₁=6.3 Hz, J₂=3.1 Hz, 1H, ═CH), 5.60 (q, J=6.7 Hz, 1H, ═CH), 2.13 (qd, J₁=7.1 Hz, J₂=3.0 Hz, 2H, CH₂), 1.53-1.41 (m, 2H, CH₂), 1.40-1.21 (m, 6H, CH₂×3), 0.87 (t, J=6.8 Hz, 3H, CH₃); ¹³C NMR (100 MHz, CDCl₃): δ=206.2, 139.2, 128.6 (q, J=32.2 Hz), 126.6, 125.4 (q, J=3.8 Hz), 124.4 (q, J=269.9 Hz), 95.7, 93.8, 31.7, 29.1, 28.9, 28.5, 22.7, 14.0; ¹⁹F NMR (376 MHz, CDCl₃) δ=−62.9; IR (neat): ν=2927, 2856, 1950, 1615, 1439, 1322, 1163, 1121, 1064 cm⁻¹; MS (70 eV, EI) m/z (%): 268 (M⁺, 1.13), 129 (100).

### Example 14

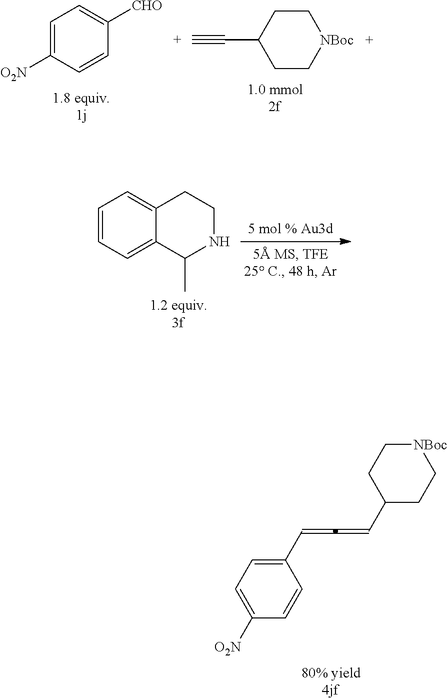

The operation is the same as Example 1. 5 Å molecular sieves (250.1 mg), Au3d (43.7 mg, 0.05 mmol), aldehyde 1j (272.0 mg, 1.8 mmol), 1-methyl-1,2,3,4-tetrahydroisoquinoline 3f (176.5 mg, 1.2 mmol) and terminal alkyne 2f (209.2 mg, 1.0 mmol) were reacted in 2,2,2-trifluoroethanol (1 mL) to afford an allene product 4jf (276.2 mg, 80% yield) (purified by flash column chromatography (eluent:petroleum ether/ethyl acetate=10/1)): oily liquid; ¹H NMR (400 MHz, CDCl₃): δ=8.16 (d, J=9.2 Hz, 2H, Ar—H), 7.40 (d, J=8.8 Hz, 2H, Ar—H), 6.27 (dd, J=6.6 Hz, J₂=3.0 Hz, 1H, ═CH), 5.71 (t, J=6.0 Hz, 1H, ═CH), 4.27-3.84 (br, 2H, CH₂), 2.82 (t, J=12.2 Hz, 2H, CH₂), 2.42-2.28 (m, 1H, CH), 1.88-1.73 (m, 2H, CH₂), 1.45 (s, 9H, OC(CH₃)₃), 1.43-1.29 (m, 2H, CH₂); ¹³C NMR (100 MHz, CDCl₃): δ=206.1, 154.6, 146.3, 142.0, 126.8, 123.9, 100.2, 95.1, 79.4, 43.4, 35.4, 31.7, 28.3; IR (neat): ν=2976, 2932, 2852, 1946, 1680, 1595, 1515, 1421, 1338, 1229, 1164, 1110 cm⁻¹; MS (ESI) m/z (%): 367 (M+Na⁺); HRMS Calcd. for C₁₉H₂₄O₄N₂Na (M+Na⁺): 367.1628, found 367.1623.

### Example 15

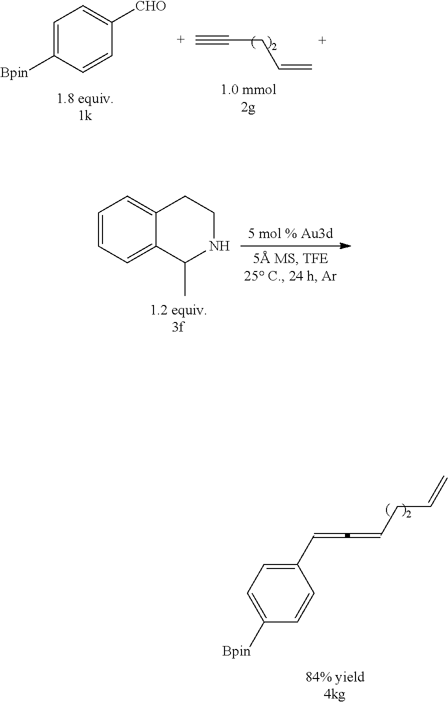

The operation is the same as Example 1. 5 Å molecular sieves (250.2 mg), Au3d (43.1 mg, 0.05 mmol), aldehyde 1k (417.5 mg, 1.8 mmol), 1-methyl-1,2,3,4-tetrahydroisoquinoline 3f (176.8 mg, 1.2 mmol) and terminal alkyne 2g (80.3 mg, 1.0 mmol) were reacted in 2,2,2-trifluoroethanol (1 mL) to afford an allene product 4 kg (249.2 mg, 84% yield) (purified by flash column chromatography (eluent: petroleum ether/ethyl acetate=200/1)): oily liquid; ¹H NMR (400 MHz, CDCl₃): δ=7.73 (d, J=8.0 Hz, 2H, Ar—H), 7.28 (d, J=8.0 Hz, 2H, Ar—H), 6.23-6.06 (m, 1H, ═CH), 5.94-5.77 (m, 1H, ═CH), 5.67-5.50 (m, 1H, ═CH), 5.05 (dd, J₁=17.0 Hz, J₂=1.4 Hz, 1H, one proton of ═CH₂), 5.00 (d, J=10.0 Hz, 1H, one proton of ═CH₂), 2.32-2.15 (m, 4H, CH₂×2), 1.34 (s, 12H, CH₃×4); ¹³C NMR (100 MHz, CDCl₃): δ=205.7, 137.9, 137.8, 135.0, 125.9, 115.2, 95.1, 94.4, 83.6, 33.1, 28.0, 24.81, 24.79; IR (neat): ν=2977, 2926, 1947, 1607, 1356, 1320, 1142, 1086 cm⁻¹; MS (FI) m/z (%): 296 (M⁺(¹¹B)); HRMS Calcd. for C₁₉H₂₅O₂¹⁰B (M⁺): 295.1978, found 295.1977.

### Example 16

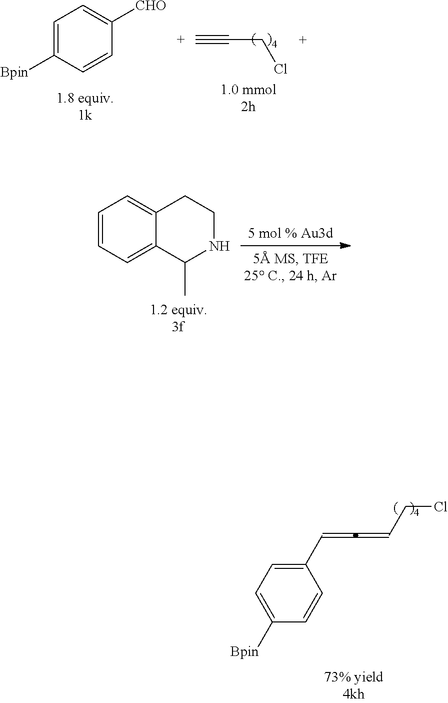

The operation is the same as Example 1. 5 Å molecular sieves (250.6 mg), Au3d (43.5 mg, 0.05 mmol), aldehyde 1k (417.6 mg, 1.8 mmol), 1-methyl-1,2,3,4-tetrahydroisoquinoline 3f (176.9 mg, 1.2 mmol) and terminal alkyne 2h (116.4 mg, 1.0 mmol) were reacted in 2,2,2-trifluoroethanol (1 mL) to afford an allene product 4kh (243.2 mg, 73% yield) (purified by flash column chromatography (eluent:petroleum ether/ethyl acetate=80/1)): oily liquid; ¹H NMR (400 MHz, CDCl₃): δ=7.74 (d, J=7.6 Hz, 2H, Ar—H), 7.27 (d, J=8.0 Hz, 2H, Ar—H), 6.15 (dt, J=6.3 Hz, J₂=3.1 Hz, 1H, ═CH), 5.57 (q, J=6.7 Hz, 1H, ═CH), 3.53 (t, J=6.6 Hz, 2H, CH₂), 2.17 (qd, J₁=6.9 Hz, J₂=2.8 Hz, 2H, CH₂), 1.91-1.79 (m, 2H, CH₂), 1.70-1.56 (m, 2H, CH₂), 1.34 (s, 12H, CH₃×4); ¹³C NMR (100 MHz, CDCl₃): δ=205.7, 137.7, 135.0, 125.8, 95.1, 94.3, 83.6, 44.6, 31.9, 27.7, 26.1, 24.8, 24.7; IR (neat): ν=2977, 2935, 1947, 1607, 1356, 1319, 1142, 1086 cm⁻¹; MS (FI) m/z (%): 332 (M⁺(¹¹B, ³⁵Cl)); HRMS Calcd. for C₁₉H₂₆O₂¹⁰B³⁵Cl (M⁺): 331.1745, found 331.1751.

### Example 17

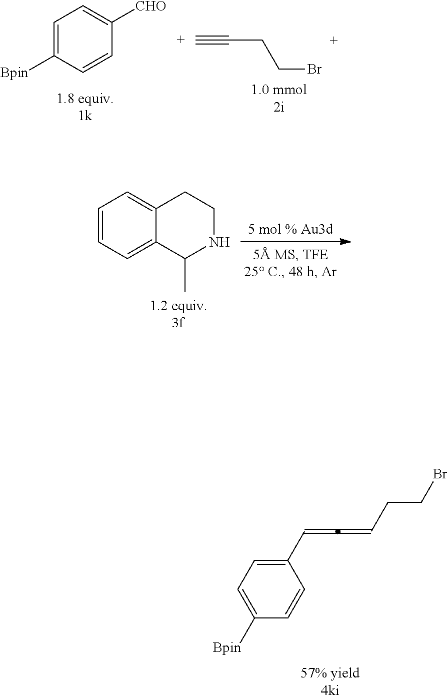

The operation is the same as Example 1. 5 Å molecular sieves (250.5 mg), Au3d (43.5 mg, 0.05 mmol), aldehyde 1k (418.1 mg, 1.8 mmol), 1-methyl-1,2,3,4-tetrahydroisoquinoline 3f (176.7 mg, 1.2 mmol) and terminal alkyne 2i (133.0 mg, 1.0 mmol) were reacted in 2,2,2-trifluoroethanol (1 mL) to afford an allene product 4ki (199.9 mg, 57% yield) (purified by flash column chromatography (eluent:petroleum ether/ethyl acetate=200/1)): oily liquid; ¹H NMR (400 MHz, CDCl₃): δ=7.74 (d, J=8.0 Hz, 2H, Ar—H), 7.31 (d, J=7.6 Hz, 2H, Ar—H), 6.22 (dt, J=6.1 Hz, J₂=3.0 Hz, 1H, ═CH), 5.62 (q, J=6.7 Hz, 1H, ═CH), 3.48 (t, J=6.8 Hz, 2H, CH₂), 2.78-2.62 (m, 2H, CH₂), 1.34 (s, 12H, CH₃×4); ¹³C NMR (100 MHz, CDCl₃): δ=206.2, 137.0, 135.0, 126.1, 95.9, 92.5, 83.6, 32.0, 31.6, 24.81, 24.78; IR (neat): ν=2977, 1949, 1607, 1393, 1356, 1320, 1269, 1142, 1085 cm⁻¹; MS (70 eV, EI) m/z (%): 347 (M⁺(¹⁰B, ⁷⁹Br), 5.98), 348 (M⁺(¹⁰B, ⁷⁹Br), 22.06), 349 (M⁺(¹⁰B, ⁸¹Br), 9.26), 350 (M⁺(¹¹B, ⁸¹Br), 20.95), 169 (100); HRMS Calcd. for C₁₇H₂₂O₂¹⁰B⁷⁹Br (M⁺): 347.0927, found 347.0931.

### Example 18

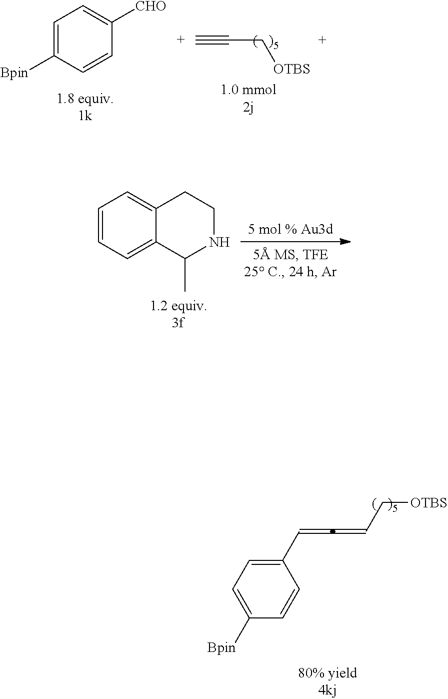

The operation is the same as Example 1. 5 Å molecular sieves (250.6 mg), Au3d (43.5 mg, 0.05 mmol), aldehyde 1k (417.2 mg, 1.8 mmol), 1-methyl-1,2,3,4-tetrahydroisoquinoline 3f (176.4 mg, 1.2 mmol) and terminal alkyne 2j (226.1 mg, 1.0 mmol) were reacted in 2,2,2-trifluoroethanol (1 mL) to afford an allene product 4kj (355.3 mg, 80% yield) (purified by flash column chromatography (eluent:petroleum ether (200 mL); petroleum ether/ethyl acetate=80/1 (405 mL))): oily liquid; ¹H NMR (400 MHz, CDCl₃): δ=7.73 (d, J=8.0 Hz, 2H, Ar—H), 7.28 (d, J=8.0 Hz, 2H, Ar—H), 6.12 (dt, J₁=6.3 Hz, J₂=3.1 Hz, 1H, ═CH), 5.58 (q, J=6.7 Hz, 1H, ═CH), 3.58 (t, J=6.6 Hz, 2H, OCH₂), 2.14 (qd, J₁=7.0 Hz, J₂=2.9 Hz, 2H, CH₂), 1.57-1.44 (m, 4H, CH₂×2), 1.44-1.36 (m, 2H, CH₂), 1.34 (s, 12H, CH₃×4), 0.89 (s, 9H, C(CH₃)₃), 0.04 (s, 6H, CH₃×2); ¹³C NMR (100 MHz, CDCl₃): δ=205.7, 138.1, 135.0, 125.9, 95.0, 94.8, 83.6, 63.2, 32.6, 28.9, 28.6, 26.0, 25.4, 24.9, 24.8, 18.4, −5.3; IR (neat): ν=2931, 2857, 1997, 1608, 1358, 1144, 1088 cm⁻¹; MS (FI) m/z (%): 442 (M⁺(¹¹B)); HRMS Calcd. for C₂₆H₄₃O₃Si¹⁰B (M⁺): 441.3105, found 441.3108.

### Example 19

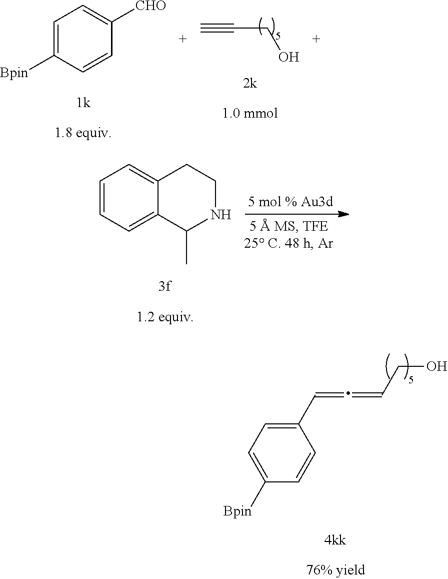

The operation is the same as Example 1. 5 Å molecular sieves (250.1 mg), Au3d (43.4 mg, 0.05 mmol), aldehyde 1k (417.7 mg, 1.8 mmol), 1-methyl-1,2,3,4-tetrahydroisoquinoline 3f (176.9 mg, 1.2 mmol) and terminal alkyne 2k (112.1 mg, 1.0 mmol) were reacted in 2,2,2-trifluoroethanol (1 mL) to afford an allene product 4kk (258.5 mg, 96% purity, 76% yield) (purified by flash column chromatography (Eluent: petroleum ether/ethyl acetate=10/1)): oily liquid; ¹H NMR (400 MHz, CDCl₃): δ=7.73 (d, J=7.6 Hz, 2H, Ar—H), 7.28 (d, J=8.0 Hz, 2H, Ar—H), 6.13 (dt, J₁=6.4 Hz, J₂=3.1 Hz, 1H, ═CH), 5.58 (q, J=6.5 Hz, 1H, ═CH), 3.62 (t, J=6.6 Hz, 2H, CH₂), 2.15 (qd, J₁=6.9 Hz, J₂=3.0 Hz, 2H, CH₂), 1.80-1.62 (brs, 1H, OH), 1.62-1.47 (m, 4H, CH₂×2), 1.47-1.38 (m, 2H, CH₂), 1.34 (s, 12H, CH₃×4); ¹³C NMR (100 MHz, CDCl₃): δ=205.6, 138.0, 134.9, 125.8, 94.8, 94.7, 83.6, 62.6, 32.3, 28.7, 28.4, 25.2, 24.73, 24.70; IR (neat): ν=3354, 2977, 2931, 2858, 1947, 1607, 1356, 1319, 1142, 1086 cm⁻¹; MS (FI) m/z (%): 328 (M⁺(¹¹B)); HRMS Calcd. for C₂₀H₂₉O₃¹⁰B (M⁺): 327.2241, found 327.2245.

### Example 20

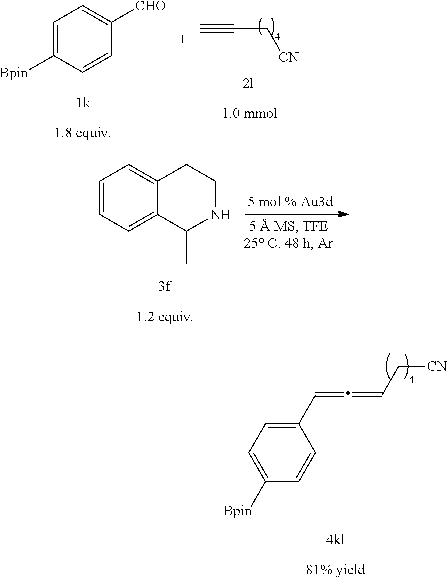

The operation is the same as Example 1. 5 Å molecular sieves (250.1 mg), Au3d (43.4 mg, 0.05 mmol), aldehyde 1k (417.9 mg, 1.8 mmol), 1-methyl-1,2,3,4-tetrahydroisoquinoline 3f (176.7 mg, 1.2 mmol) and terminal alkyne 21 (107.0 mg, 1.0 mmol) were reacted in 2,2,2-trifluoroethanol (1 mL) to afford an allene product 4kl (262.8 mg, 81% yield) (purified by flash column chromatography (eluent:petroleum ether/ethyl acetate=40/1 (410 mL); petroleum ether/ethyl acetate=20/1 (420 mL)): oily liquid; ¹H NMR (400 MHz, CDCl₃): δ=7.74 (d, J=8.0 Hz, 2H, Ar—H), 7.27 (d, J=8.0 Hz, 2H, Ar—H), 6.17 (dt, J₁=6.1 Hz, J₂=3.0 Hz, 1H, ═CH), 5.57 (q, J=6.5 Hz, 1H, ═CH), 2.33 (t, J=7.0 Hz, 2H, CH₂), 2.18 (qd, J₁=7.0 Hz, J₂=3.0 Hz, 2H, CH₂), 1.80-1.69 (m, 2H, CH₂), 1.69-1.58 (m, 2H, CH₂), 1.34 (s, 12H, CH₃×4); ¹³C NMR (100 MHz, CDCl₃): δ=205.7, 137.6, 135.0, 125.8, 119.5, 95.3, 94.0, 83.7, 27.7, 27.6, 24.81, 24.78, 24.73, 16.9; IR (neat): ν=2975, 2937, 2896, 1949, 1605, 1355, 1323, 1147, 1089 cm⁻¹; MS (FI) m/z (%): 323 (M⁺(¹¹B)); HRMS Calcd. for C₂₀H₂₆O₂N¹⁰B (M⁺): 322.2087, found 322.2081.

### Example 21

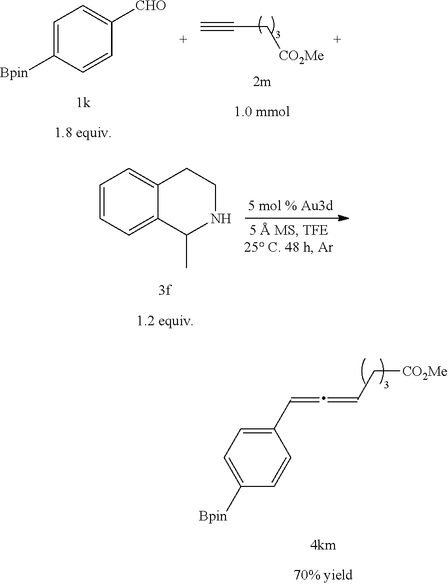

The operation is the same as Example 1. 5 Å molecular sieves (250.1 mg), Au3d (43.7 mg, 0.05 mmol), aldehyde 1k (418.0 mg, 1.8 mmol), 1-methyl-1,2,3,4-tetrahydroisoquinoline 3f (176.5 mg, 1.2 mmol) and terminal alkyne 2m (126.1 mg, 1.0 mmol) were reacted in 2,2,2-trifluoroethanol (1 mL) to afford an allene product 4 km (240.6 mg, 70% yield) (purified by flash column chromatography (eluent:petroleum ether/ethyl acetate=80/1 (405 mL); petroleum ether/ethyl acetate=40/1 (820 mL)): oily liquid; ¹H NMR (400 MHz, CDCl₃): δ=7.73 (d, J=8.0 Hz, 2H, Ar—H), 7.27 (d, J=9.2 Hz, 2H, Ar—H), 6.16 (dt, J₁=6.1 Hz, J₂=3.0 Hz, 1H, ═CH), 5.57 (q, J=6.7 Hz, 1H, ═CH), 3.66 (s, 3H, OCH₃), 2.39 (t, J=7.6 Hz, 2H, CH₂), 2.25-2.12 (m, 2H, CH₂), 1.89-1.75 (m, 2H, CH₂), 1.34 (s, 12H, CH₃×4); ¹³C NMR (100 MHz, CDCl₃): δ=205.8, 173.8, 137.7, 135.0, 125.9, 95.2, 94.1, 83.6, 51.4, 33.3, 27.9, 24.80, 24.77, 24.1; IR (neat): ν=2977, 1947, 1737, 1607, 1356, 1142, 1086 cm⁻¹; MS (FI) m/z (%): 342 (M⁺(¹¹B)); HRMS Calcd. for C₂₀H₂₇O₄¹⁰B (M⁺): 341.2033, found 341.2039.

### Example 22

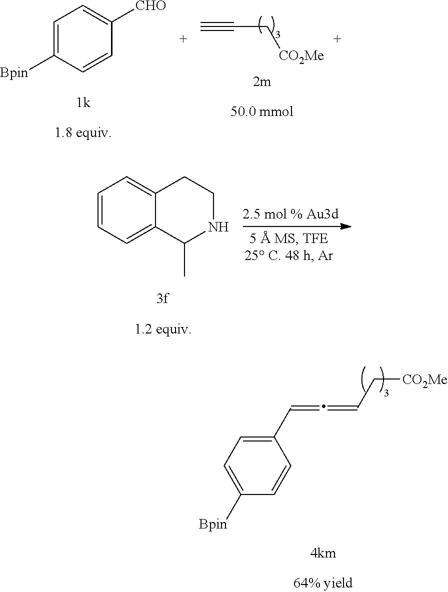

The operation is the same as Example 1. 5 Å molecular sieves (12.5025 g), Au3d (1.0851 g, 1.25 mmol), aldehyde 1k (20.8894 g, 90 mmol), 1-methyl-1,2,3,4-tetrahydroisoquinoline 3f (8.8317 g, 60 mmol) and terminal alkyne 2m (6.3093 g, 50 mmol) were reacted in 2,2,2-trifluoroethanol (50 mL) to afford an allene product 4 km (10.9863 g, 64% yield) (purified by flash column chromatography (eluent:petroleum ether/ethyl acetate=40/1)): oily liquid; ¹H NMR (400 MHz, CDCl₃): δ=7.73 (d, J=8.0 Hz, 2H, Ar—H), 7.27 (d, J=9.2 Hz, 2H, Ar—H), 6.16 (dt, J=6.1 Hz, J₂=3.0 Hz, 1H, ═CH), 5.57 (q, J=6.7 Hz, 1H, ═CH), 3.66 (s, 3H, OCH₃), 2.39 (t, J=7.4 Hz, 2H, CH₂), 2.18 (qd, J₁=6.9 Hz, J₂=2.9 Hz, 2H, CH₂), 1.89-1.74 (m, 2H, CH₂), 1.34 (s, 12H, CH₃×4).

### Example 23

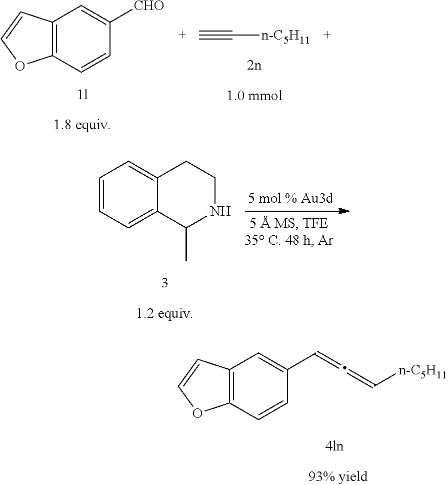

The operation is the same as Example 1. 5 Å molecular sieves (250.4 mg), Au3d (43.5 mg, 0.05 mmol), aldehyde 11 (263.5 mg, 1.8 mmol), 1-methyl-1,2,3,4-tetrahydroisoquinoline 3f (177.1 mg, 1.2 mmol) and terminal alkyne 2n (96.0 mg, 1.0 mmol) were reacted in 2,2,2-trifluoroethanol (1 mL) to afford an allene product 4ln (208.9 mg, 93% yield) (purified by flash column chromatography (eluent:petroleum ether)): oily liquid; ¹H NMR (400 MHz, CDCl₃): δ=7.59 (d, J=2.4 Hz, 1H, Ar—H), 7.48 (s, 1H, Ar—H), 7.42 (d, J=8.8 Hz, 1H, Ar—H), 7.31-7.22 (m, 1H, Ar—H), 6.75-6.68 (m, 1H, Ar—H), 6.22 (dt, J₁=6.3 Hz, J₂=3.1 Hz, 1H, ═CH), 5.58 (q, J=6.7 Hz, 1H, ═CH), 2.14 (qd, J₁=7.1 Hz, J₂=2.9 Hz, 2H, CH₂), 1.54-1.43 (m, 2H, CH₂), 1.42-1.23 (m, 4H, CH₂×2), 0.89 (t, J=7.0 Hz, 3H, CH₃); ¹³C NMR (100 MHz, CDCl₃): δ=204.7, 154.1, 145.2, 129.9, 127.8, 123.1, 118.9, 111.3, 106.4, 95.1, 94.6, 31.4, 28.8, 22.4, 14.0; IR (neat): ν=2925, 2855, 1948, 1466, 1452, 1262, 1192, 1111, 1031 cm⁻¹; MS (70 eV, EI) m/z (%): 226 (M⁺, 12.48), 170 (100); HRMS Calcd. for C₁₆H₁₈O (M⁺): 226.1352, found 226.1356.

### Example 24

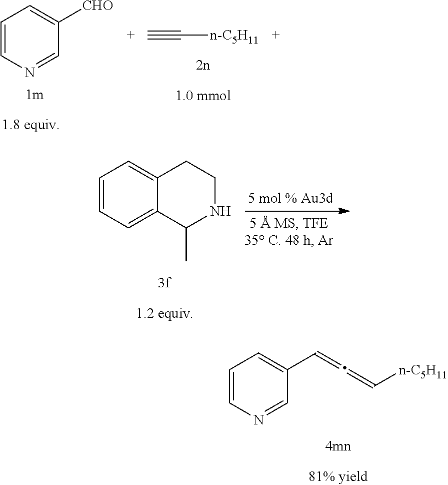

The operation is the same as Example 1. 5 Å molecular sieves (250.4 mg), Au3d (43.1 mg, 0.05 mmol), aldehyde 1m (193.1 mg, 1.8 mmol), 1-methyl-1,2,3,4-tetrahydroisoquinoline 3f (176.5 mg, 1.2 mmol) and terminal alkyne 2n (96.0 mg, 1.0 mmol) were reacted in 2,2,2-trifluoroethanol (1 mL) to afford an allene product 4mn (152.3 mg, 81% yield) (purified by flash column chromatography (eluent:petroleum ether/ethyl acetate=20/1)): oily liquid; ¹H NMR (400 MHz, CDCl₃): δ=8.51 (d, J=1.6 Hz, 1H, Ar—H), 8.41 (dd, J₁=4.8 Hz, J₂=1.2 Hz, 1H, Ar—H), 7.58 (dt, J₁=7.6 Hz, J₂=1.6 Hz, 1H, Ar—H), 7.21 (dd, J₁=7.6 Hz, J₂=4.8 Hz, 1H, Ar—H), 6.10 (dt, J₁=6.4 Hz, J₂=3.2 Hz, 1H, ═CH), 5.63 (q, J=6.7 Hz, 1H, ═CH), 2.14 (qd, J₁=7.2 Hz, J₂=3.1 Hz, 2H, CH₂), 1.57-1.41 (m, 2H, CH₂), 1.40-1.22 (m, 4H, CH₂×2), 0.89 (t, J=7.0 Hz, 3H, CH₃); ¹³C NMR (100 MHz, CDCl₃): δ=205.4, 147.9, 147.5, 133.1, 130.9, 123.2, 95.6, 91.2, 31.2, 28.6, 28.3, 22.3, 13.9; IR (neat): ν=2925, 2855, 1949, 1570, 1480, 1444, 1390, 1180, 1120, 1023 cm⁻¹; MS (70 eV, EI) m/z (%): 187 (M⁺, 1.42), 130 (100); HRMS Calcd. for C₁₃H₁₇N (M⁺): 187.1356, found 187.1359.

### Example 25

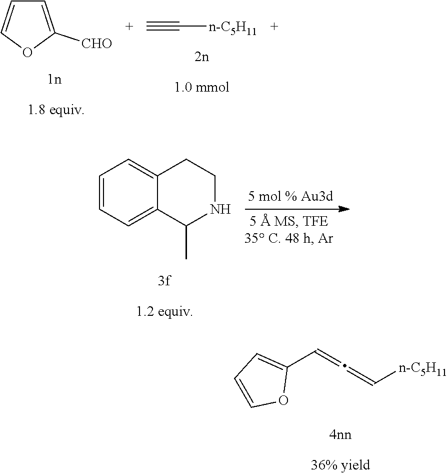

The operation is the same as Example 1. 5 Å molecular sieves (250.2 mg), Au3d (43.5 mg, 0.05 mmol), aldehyde in (173.4 mg, 1.8 mmol), 1-methyl-1,2,3,4-tetrahydroisoquinoline 3f (176.5 mg, 1.2 mmol) and terminal alkyne 2n (96.3 mg, 1.0 mmol) were reacted in 2,2,2-trifluoroethanol (1 mL) to afford an allene product 4nn (63.8 mg, 36% yield) (purified by flash column chromatography (eluent:petroleum ether)): oily liquid; ¹H NMR (400 MHz, CDCl₃): δ=7.38-7.31 (m, 1H, Ar—H), 6.37 (dd, J₁=3.2 Hz, J₂=2.0 Hz, 1H, Ar—H), 6.18 (d, J=3.2 Hz, 1H, Ar—H), 6.10 (dt, J₁=6.3 Hz, J₂=3.1 Hz, 1H, ═CH), 5.60 (q, J=6.8 Hz, 1H, ═CH), 2.12 (qd, J₁=7.1 Hz, J₂=3.0 Hz, 2H, CH₂), 1.53-1.42 (m, 2H, CH₂), 1.40-1.22 (m, 4H, CH₂×2), 0.89 (t, J=7.0 Hz, 3H, CH₃); ¹³C NMR (100 MHz, CDCl₃): δ=204.4, 149.0, 141.7, 111.3, 106.5, 95.6, 85.3, 31.3, 28.8, 28.5, 22.4, 14.0; IR (neat): ν=2926, 2856, 1950, 1462, 1249, 1174, 1150, 1076, 1010 cm⁻¹; MS (70 eV, EI) m/z (%): 176 (M⁺, 11.56), 120 (100); HRMS Calcd. for C₁₂H₁₆O (M⁺): 176.1196, found 176.1200.

### Example 26

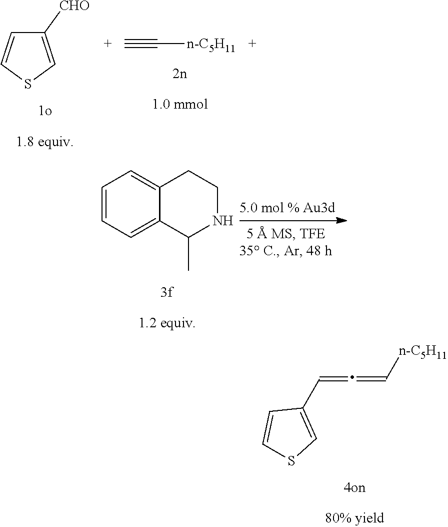

The operation is the same as Example 1. 5 Å molecular sieves (250.5 mg), Au3d (43.4 mg, 0.05 mmol), aldehyde to (201.7 mg, 1.8 mmol), 1-methyl-1,2,3,4-tetrahydroisoquinoline 3f (176.6 mg, 1.2 mmol) and terminal alkyne 2n (96.3 mg, 1.0 mmol) were reacted in 2,2,2-trifluoroethanol (1 mL) to afford an allene product 4on (154.0 mg, 80% yield) (purified by flash column chromatography (eluent:petroleum ether)): oily liquid; ¹H NMR (400 MHz, CDCl₃): δ=7.24 (dd, J₁=5.2 Hz, J₂=2.8 Hz, 1H, Ar—H), 7.06 (d, J=4.8 Hz, 1H, Ar—H), 7.03 (d, J=2.8 Hz, 1H, Ar—H), 6.18 (dt, J₁=6.1 Hz, J₂=3.1 Hz, 1H, ═CH), 5.49 (q, J=6.5 Hz, 1H, ═CH), 2.10 (qd, J₁=7.1 Hz, J₂=3.0 Hz, 2H, CH₂), 1.52-1.42 (m, 2H, CH₂), 1.39-1.24 (m, 4H, CH₂×2), 0.89 (t, J=7.0 Hz, 3H, CH₃); ¹³C NMR (100 MHz, CDCl₃): δ=205.4, 136.5, 126.2, 125.6, 120.0, 94.2, 89.1, 31.3, 28.78, 28.77, 22.4, 14.0; IR (neat): ν=2924, 2854, 1950, 1462, 1378, 1261, 1232, 1077 cm⁻¹; MS (70 eV, EI) m/z (%): 192 (M⁺, 5.72), 135 (100); HRMS Calcd. for C₁₂H₁₆S (M⁺): 192.0967, found 192.0966.

### Example 27

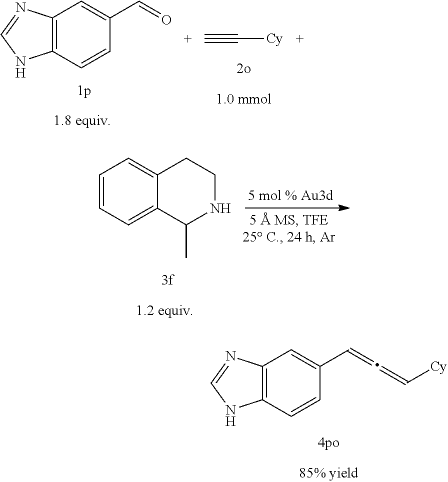

The operation is the same as Example 1. 5 Å molecular sieves (250.6 mg), Au3d (43.5 mg, 0.05 mmol), aldehyde 1p (263.4 mg, 1.8 mmol), 1-methyl-1,2,3,4-tetrahydroisoquinoline 3f (176.5 mg, 1.2 mmol) and terminal alkyne 2o (108.3 mg, 1.0 mmol) were reacted in 2,2,2-trifluoroethanol (1 mL) to afford an allene product 4po (203.0 mg, 85% yield) (purified by flash column chromatography (eluent:petroleum ether/ethyl acetate=4/1)): white solid; m.p. 152.0-152.9° C. (recrystallized from ethyl acetate); ¹H NMR (400 MHz, CDCl₃): δ=10.80-10.04 (brs, 1H, NH), 8.03 (s, 1H, Ar—H), 7.59 (s, 1H, Ar—H), 7.43 (s, 2H, Ar—H), 6.28 (dd, J₁=6.4 Hz, J₂=2.8 Hz, 1H, ═CH), 5.60 (t, J=6.0 Hz, 1H, ═CH), 2.23-2.04 (m, 1H, CH), 1.94-1.82 (m, 2H), 1.82-1.68 (m, 2H), 1.68-1.57 (m, 1H), 1.40-1.08 (m, 5H); ¹³C NMR (100 MHz, CDCl₃): δ=203.7, 139.4, 134.7, 128.4, 126.0, 123.7, 118.1, 109.9, 101.2, 95.5, 37.7, 33.2, 33.1, 26.1, 26.02, 26.01; IR (neat): ν=3139, 2918, 2848, 1946, 1623, 1506, 1442, 1341, 1307, 1081 cm⁻¹; MS (ESI) m/z (%): 239 (M+H⁺); Anal. Calcd. for C₁₆H₁₈N₂: C 80.63, H, 7.61, N 11.75, found C 80.49, H 7.69, N 11.72.

### Example 28

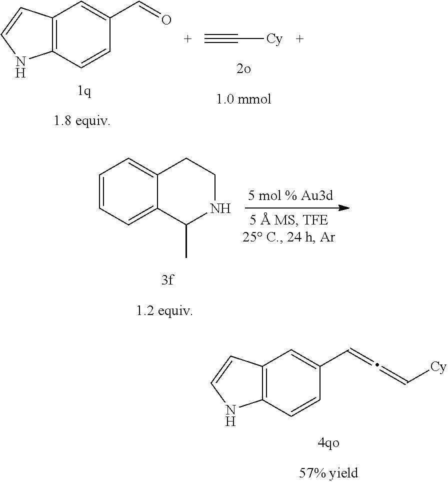

The operation is the same as Example 1. 5 Å molecular sieves (250.2 mg), Au3d (43.1 mg, 0.05 mmol), aldehyde 1q (261.0 mg, 1.8 mmol), 1-methyl-1,2,3,4-tetrahydroisoquinoline 3f (176.4 mg, 1.2 mmol) and terminal alkyne 2o (107.8 mg, 1.0 mmol) were reacted in 2,2,2-trifluoroethanol (1 mL) to afford an allene product 4qo (133.6 mg, 57% yield) (purified by flash column chromatography (eluent:petroleum ether/ethyl acetate=40/1)): oily liquid; ¹H NMR (400 MHz, CDCl₃): δ=8.22-7.85 (brs, 1H, NH), 7.52 (s, 1H, Ar—H), 7.29 (d, J=8.4 Hz, 1H, Ar—H), 7.22 (t, J=7.6 Hz, 1H, Ar—H) 7.13 (s, 1H, Ar—H), 6.49 (s, 1H, Ar—H), 6.28 (dd, J₁=6.0 Hz, J₂=2.8 Hz, 1H, ═CH), 5.56 (t, J=6.2 Hz, 1H, ═CH), 2.22-2.05 (m, 1H, CH), 1.95-1.80 (m, 2H), 1.80-1.68 (m, 2H), 1.68-1.58 (m, 1H), 1.38-1.08 (m, 5H); ¹³C NMR (100 MHz, CDCl₃): δ=203.3, 134.9, 128.1, 126.7, 124.5, 120.9, 118.5, 111.2, 102.4, 100.8, 96.1, 37.7, 33.2, 33.1, 26.1, 26.03, 26.02; IR (neat): ν=3410, 2921, 2848, 1945, 1447, 1415, 1319, 1285, 1091 cm⁻¹; MS (ESI) m/z (%): 238 (M+H⁺); HRMS Calcd. for C₁₇H₂₀N (M+H⁺): 238.1590, found 238.1588.

### Example 29

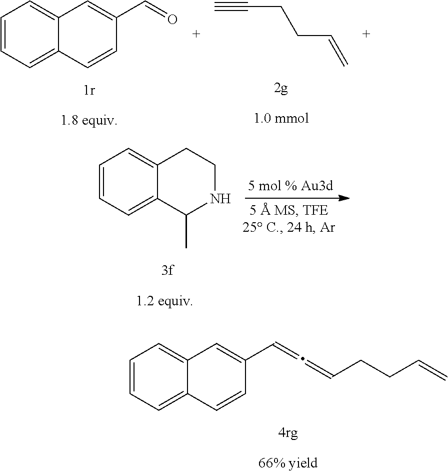

The operation is the same as Example 1. 5 Å molecular sieves (250.4 mg), Au3d (43.7 mg, 0.05 mmol), aldehyde 1r (281.5 mg, 1.8 mmol), 1-methyl-1,2,3,4-tetrahydroisoquinoline 3f (176.4 mg, 1.2 mmol) and terminal alkyne 2g (80.0 mg, 1.0 mmol) were reacted in 2,2,2-trifluoroethanol (1 mL) to afford an allene product 4rg (145.7 mg, 66% yield) (purified by flash column chromatography (eluent: petroleum ether)): oily liquid; ¹H NMR (400 MHz, CDCl₃): δ=7.82-7.70 (m, 3H, Ar—H), 7.64 (s, 1H, Ar—H), 7.53-7.47 (m, 1H, Ar—H), 7.47-7.36 (m, 2H, Ar—H), 6.37-6.26 (m, 1H, ═CH), 5.96-5.78 (m, 1H, ═CH), 5.65 (q, J=6.1 Hz, 1H, ═CH), 5.08 (d, J=17.6 Hz, 1H, one proton of ═CH₂), 5.02 (d, J=10.0 Hz, 1H, one proton of ═CH₂), 2.36-2.17 (m, 4H, CH₂×2); ¹³C NMR (100 MHz, CDCl₃): δ=205.7, 137.8, 133.7, 132.5, 132.4, 128.1, 127.63, 127.58, 126.1, 125.4, 125.3, 124.6, 115.2, 95.3, 94.6, 33.1, 28.1; IR (neat): ν=3054, 2976, 2909, 2843, 1945, 1639, 1597, 1507, 1435, 1264 cm⁻¹; MS (70 eV, EI) m/z (%): 220 (M⁺, 91.99), 178 (100); HRMS Calcd. for C₁₇H₁₆ (M⁺): 220.1247, found 220.1248.

### Example 30

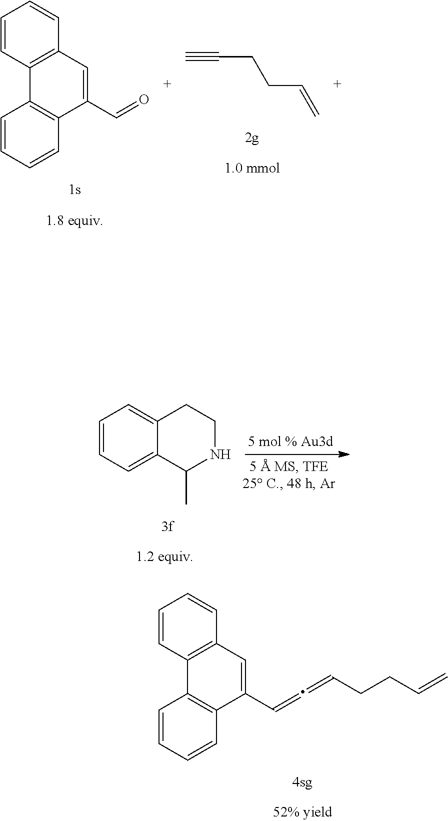

The operation is the same as Example 1. 5 Å molecular sieves (250.4 mg), Au3d (43.5 mg, 0.05 mmol), aldehyde is (371.4 mg, 1.8 mmol), 1-methyl-1,2,3,4-tetrahydroisoquinoline 3f (176.8 mg, 1.2 mmol) and terminal alkyne 2g (80.1 mg, 1.0 mmol) were reacted in 2,2,2-trifluoroethanol (1 mL) to afford an allene product 4sg (139.9 mg, 52% yield) (purified by flash column chromatography (eluent: petroleum ether)): oily liquid; ¹H NMR (400 MHz, CDCl₃): δ=8.74-8.66 (m, 1H, Ar—H), 8.62 (d, J=8.0 Hz, 1H, Ar—H), 8.36-8.27 (m, 1H, Ar—H), 7.87-7.81 (m, 1H, Ar—H), 7.78 (s, 1H, Ar—H), 7.69-7.48 (m, 4H, Ar—H), 6.87-6.78 (m, 1H, ═CH), 5.96-5.80 (m, 1H, ═CH), 5.64 (q, J=6.5 Hz, 1H, ═CH), 5.08 (d, J=17.2 Hz, 1H, one proton of ═CH₂), 5.01 (d, J=10.0 Hz, 1H, one proton of ═CH₂), 2.39-2.22 (m, 4H, CH₂×2); ¹³C NMR (100 MHz, CDCl₃): δ=206.7, 137.9, 131.8, 130.7, 130.2, 129.8, 129.6, 128.3, 126.7, 126.5, 126.34, 126.28, 125.9, 124.4, 123.1, 122.5, 115.3, 93.2, 91.7, 33.2, 28.2; IR (neat): ν=3073, 2973, 2909, 2844, 1946, 1639, 1494, 1428, 1243, 1114 cm⁻¹; MS (70 eV, EI) m/z (%): 270 (M⁺, 100); HRMS Calcd. for C₂₁H₁₈ (M⁺): 270.1403, found 270.1408.

### Example 31

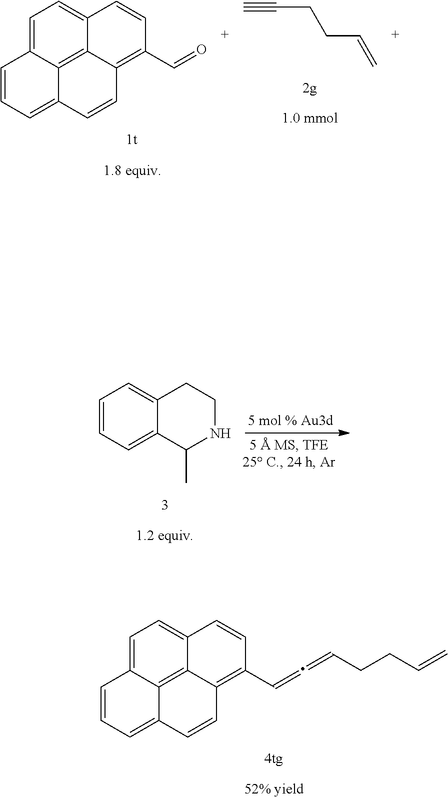

The operation is the same as Example 1. 5 Å molecular sieves (250.2 mg), Au3d (43.4 mg, 0.05 mmol), aldehyde it (414.6 mg, 1.8 mmol), 1-methyl-1,2,3,4-tetrahydroisoquinoline 3f (176.8 mg, 1.2 mmol) and terminal alkyne 2g (79.8 mg, 1.0 mmol) were reacted in 2,2,2-trifluoroethanol (1 mL) to afford an allene product 4tg (151.6 mg, 52% yield) (purified by flash column chromatography (eluent: petroleum ether)): oily liquid; ¹H NMR (400 MHz, CDCl₃): δ=8.40 (d, J=9.2 Hz, 1H, Ar—H), 8.18-8.04 (m, 5H, Ar—H), 8.04-7.94 (m, 3H, Ar—H), 7.23-7.10 (m, 1H, ═CH), 5.98-5.83 (m, 1H, ═CH), 5.75 (q, J=6.1 Hz, 1H, ═CH), 5.10 (d, J=16.8 Hz, 1H, one proton of ═CH₂), 5.03 (d, J=10.8 Hz, 1H, one proton of ═CH₂), 2.43-2.24 (m, 4H, CH₂×2); ¹³C NMR (100 MHz, CDCl₃): δ=207.2, 137.9, 131.4, 130.8, 130.1, 128.5, 127.5, 127.4, 127.3, 126.9, 125.8, 125.2, 125.1, 125.04, 124.96, 124.9, 124.7, 122.7, 115.3, 94.0, 92.0, 33.3, 28.2; IR (neat): ν=3040, 2973, 2906, 2842, 1941, 1639, 1599, 1435, 1269, 1182 cm⁻¹; MS (70 eV, EI) m/z (%): 294 (M⁺, 94.55), 253 (100); HRMS Calcd. for C₂₃H₁₈ (M⁺): 294.1403, found 294.1407.

### Example 32

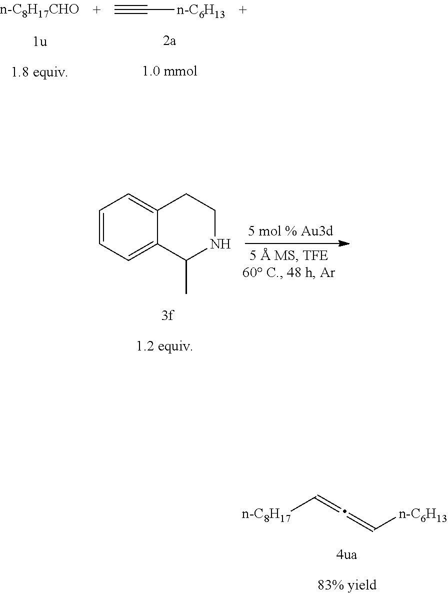

The operation is the same as Example 1. 5 Å molecular sieves (250.4 mg), Au3d (43.5 mg, 0.05 mmol), aldehyde 1u (256.5 mg, 1.8 mmol), 1-methyl-1,2,3,4-tetrahydroisoquinoline 3f (176.6 mg, 1.2 mmol) and terminal alkyne 2a (110.2 mg, 1.0 mmol) were reacted in 2,2,2-trifluoroethanol (5 mL) to afford an allene product 4ua (195.7 mg, 83% yield) (purified by flash column chromatography (eluent:petroleum ether)): oily liquid; ¹H NMR (400 MHz, CDCl₃): δ=5.06 (quintet, J=4.8 Hz, 2H, ═CH×2), 2.07-1.86 (m, 4H, CH₂×2), 1.48-1.13 (m, 20H, CH₂×10), 0.96-0.77 (m, 6H, CH₃×2); ¹³C NMR (150 MHz, CDCl₃): δ=204.0, 90.87, 90.86, 32.0, 31.8, 29.6, 29.4, 29.32, 29.28, 29.2, 29.084, 29.078, 28.9, 22.75, 22.73, 14.09, 14.08; IR (neat): ν=2957, 2922, 2853, 1963, 1462, 1378, 1261 cm⁻¹; MS (FI) m z (%): 236 (M⁺); HRMS Calcd. for C₁₇H₃₂ (M⁺): 236.2499, found 236.2500.

### Example 33

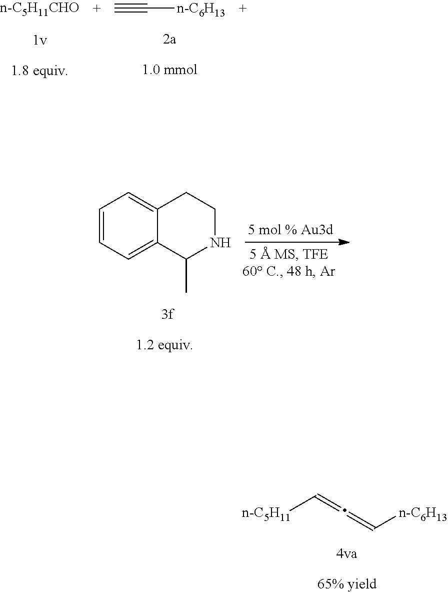

The operation is the same as Example 1. 5 Å molecular sieves (250.2 mg), Au3d (43.3 mg, 0.05 mmol), aldehyde 1v (180.5 mg, 1.8 mmol), 1-methyl-1,2,3,4-tetrahydroisoquinoline 3f (176.8 mg, 1.2 mmol) and terminal alkyne 2a (110.2 mg, 1.0 mmol) were reacted in 2,2,2-trifluoroethanol (5 mL) to afford an allene product 4va (126.1 mg, 65% yield) (purified by flash column chromatography (eluent:petroleum ether)): oily liquid; ¹H NMR (400 MHz, CDCl₃): δ=5.06 (quintet, J=4.8 Hz, 2H, ═CH×2), 2.06-1.86 (m, 4H, CH₂×2), 1.48-1.16 (m, 14H, CH₂×7), 0.97-0.74 (m, 6H, CH₃×2); ¹³C NMR (100 MHz, CDCl₃): δ=203.8, 90.9, 31.8, 31.4, 29.2, 29.04, 29.00, 28.9, 28.8, 22.7, 22.5, 14.08, 14.07; IR (neat): ν=2957, 2924, 2855, 1962, 1462, 1378, 1261, 1104, 1018 cm⁻¹; MS (FI) m/z (%): 194 (M⁺); HRMS Calcd. for C₁₄H₂₆ (M⁺): 194.2029, found 194.2032.

### Example 34

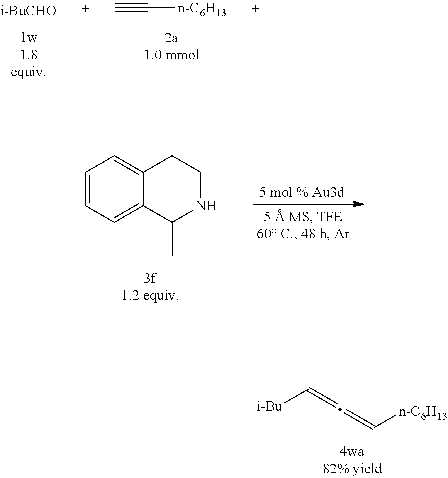

The operation is the same as Example 1. 5 Å molecular sieves (250.2 mg), Au3d (43.5 mg, 0.05 mmol), aldehyde 1w (155.3 mg, 1.8 mmol), 1-methyl-1,2,3,4-tetrahydroisoquinoline 3f (176.2 mg, 1.2 mmol) and terminal alkyne 2a (110.2 mg, 1.0 mmol) were reacted in 2,2,2-trifluoroethanol (5 mL) to afford an allene product 4wa (148.0 mg, 82% yield) (purified by flash column chromatography (eluent:petroleum ether)): oily liquid; ¹H NMR (400 MHz, CDCl₃): δ=5.11-4.94 (m, 2H, ═CH×2), 2.02-1.92 (m, 2H, CH₂), 1.92-1.78 (m, 2H, CH₂), 1.72-1.58 (m, 1H, CH), 1.43-1.19 (m, 8H, CH₂×4), 1.02-0.78 (m, 9H, CH₃×3); ¹³C NMR (100 MHz, CDCl₃): δ=204.5, 90.2, 89.4, 38.6, 31.7, 29.2, 29.0, 28.8, 28.5, 22.7, 22.3, 22.2, 14.1; IR (neat): ν=2956, 2925, 2855, 1963, 1464, 1381, 1366, 1261, 1105 cm⁻¹; MS (FI) m z (%): 180 (M⁺); HRMS Calcd. for C₁₃H₂₄ (M⁺): 180.1873, found 180.1875.

### Example 35

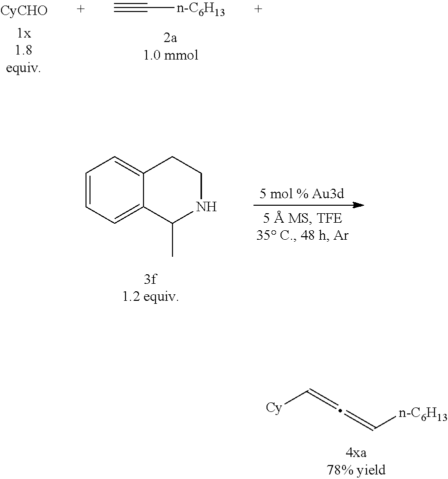

The operation is the same as Example 1. 5 Å molecular sieves (250.1 mg), Au3d (43.5 mg, 0.05 mmol), aldehyde 1x (202.5 mg, 1.8 mmol), 1-methyl-1,2,3,4-tetrahydroisoquinoline 3f (176.3 mg, 1.2 mmol) and the terminal alkyne 2a (110.0 mg, 1.0 mmol) were reacted in 2,2,2-trifluoroethanol (2 mL) to afford an allene product 4xa (161.2 mg, 78% yield) (purified by flash column chromatography (eluent:petroleum ether)): oily liquid; ¹H NMR (400 MHz, CDCl₃): δ=5.20-4.97 (m, 2H, ═CH×2), 2.06-1.86 (m, 3H, CH₂ and CH), 1.82-1.67 (m, 4H), 1.67-1.58 (m, 1H), 1.46-1.00 (m, 13H), 0.89 (t, J=6.8 Hz, 3H, CH₃); ¹³C NMR (150 MHz, CDCl₃): δ=202.7, 97.0, 91.8, 37.3, 33.18, 33.15, 31.7, 29.2, 29.1, 28.9, 26.2, 26.10, 26.09, 22.7, 14.1; IR (neat): ν=2922, 2851, 1960, 1448, 1260 cm⁻¹; MS (70 eV, EI) m/z (%): 206 (M⁺, 2.43), 136 (100); HRMS Calcd. for C₁₅H₂₆ (M⁺): 206.2029, found 206.2032.

### Example 36

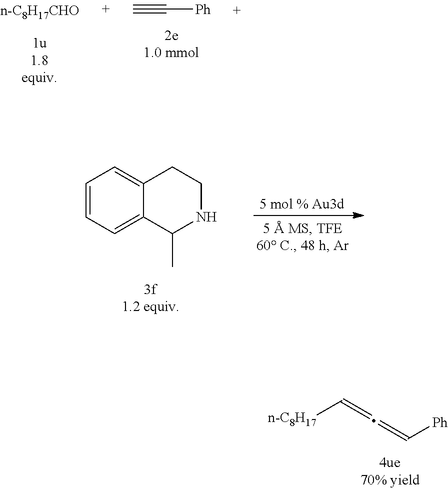

The operation is the same as Example 1. 5 Å molecular sieves (250.5 mg), Au3d (43.4 mg, 0.05 mmol), aldehyde 1u (256.1 mg, 1.8 mmol), 1-methyl-1,2,3,4-tetrahydroisoquinoline 3f (176.9 mg, 1.2 mmol) and terminal alkyne 2e (102.0 mg, 1.0 mmol) were reacted in 2,2,2-trifluoroethanol (5 mL) to afford an allene product 4ue (161.1 mg, 70% yield, 99% purity) (purified by flash column chromatography (Eluant:petroleum ether)): oily liquid; ¹H NMR (400 MHz, CDCl₃): δ=7.36-7.26 (m, 4H, Ar—H), 7.22-7.13 (m, 1H, Ar—H), 6.12 (dt, J₁=6.0 Hz, J₂=2.9 Hz, 1H, ═CH), 5.56 (q, J=6.7 Hz, 1H, ═CH), 2.12 (qd, J₁=7.1 Hz, J₂=2.7 Hz, 2H, CH₂), 1.53-1.41 (m, 2H, CH₂), 1.41-1.11 (m, 10H, CH₂×5), 0.88 (t, J=6.6 Hz, 3H, CH₃); ¹³C NMR (100 MHz, CDCl₃): δ=205.1, 135.1, 128.5, 126.54, 126.52, 95.1, 94.5, 31.8, 29.4, 29.3, 29.2, 29.1, 28.7, 22.7, 14.1; IR (neat): ν=2923, 2853, 1949, 1598, 1494, 1459 cm⁻¹; MS (FI) m/z (%): 228 (M⁺); HRMS Calcd. for C₁₇H₂₄ (M⁺): 228.1873, found 228.1869.

### Example 37

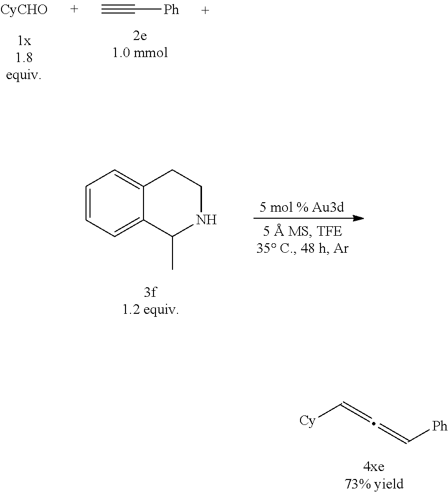

The operation is the same as Example 1. 5 Å molecular sieves (250.0 mg), Au3d (43.4 mg, 0.05 mmol), aldehyde 1x (202.5 mg, 1.8 mmol), 1-methyl-1,2,3,4-tetrahydroisoquinoline 3f (176.3 mg, 1.2 mmol) and terminal alkyne 2e (102.0 mg, 1.0 mmol) were reacted in 2,2,2-trifluoroethanol (2 mL) to afford an allene product 4xe (145.4 mg, 73% yield) (purified by flash column chromatography (eluent:petroleum ether)): oily liquid; ¹H NMR (400 MHz, CDCl₃): δ=7.36-7.26 (m, 4H, Ar—H), 7.22-7.11 (m, 1H, Ar—H), 6.15 (dd, J₁=6.2 Hz, J₂=3.0 Hz, 1H, ═CH), 5.56 (t, J=6.2 Hz, 1H, ═CH), 2.18-2.04 (m, 1H, CH), 1.92-1.78 (m, 2H), 1.78-1.68 (m, 2H), 1.68-1.57 (m, 1H), 1.37-1.08 (m, 5H); ¹³C NMR (100 MHz, CDCl₃): δ=204.0, 135.2, 128.5, 126.5, 126.4, 101.0, 95.4, 37.6, 33.15, 33.09, 26.09, 26.08, 26.02; IR (neat): ν=2921, 2849, 1946, 1597, 1493, 1446 cm⁻¹; MS (70 eV, EI) m/z (%): 198 (M⁺, 5.19), 105 (100).

### Example 38

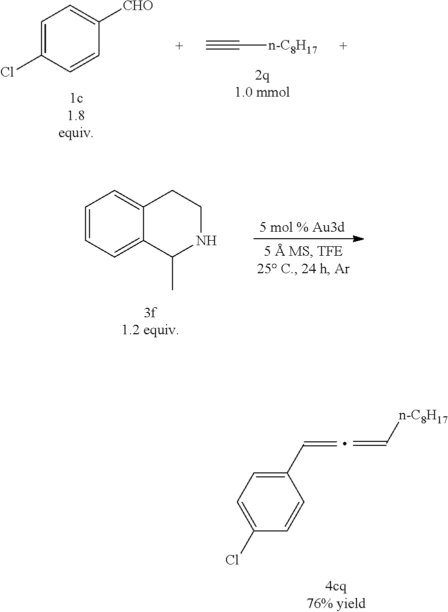

The operation is the same as Example 1. 5 Å molecular sieves (250.2 mg), Au3d (43.4 mg, 0.05 mmol), aldehyde 1c (253.4 mg, 1.8 mmol), 1-methyl-1,2,3,4-tetrahydroisoquinoline 3f (176.7 mg, 1.2 mmol) and terminal alkyne 2q (138.1 mg, 1.0 mmol) were reacted in 2,2,2-trifluoroethanol (1 mL) to afford an allene product 4cq (200.1 mg, 76% yield) (purified by flash column chromatography (eluent:petroleum ether)): oily liquid; ¹H NMR (400 MHz, CDCl₃): δ=7.28-7.22 (m, 2H, Ar—H), 7.22-7.16 (m, 2H, Ar—H), 6.07 (dt, J₁=6.3 Hz, J₂=3.1 Hz, 1H, ═CH), 5.57 (q, J=6.7 Hz, 1H, ═CH), 2.12 (qd, J₁=7.1 Hz, J₂=2.9 Hz, 2H, CH₂), 1.53-1.41 (m, 2H, CH₂), 1.41-1.14 (m, 10H, CH₂×5), 0.87 (t, J=6.8 Hz, 3H, CH₃); ¹³C NMR (100 MHz, CDCl₃): δ=205.2, 133.7, 132.1, 128.6, 127.7, 95.5, 93.7, 31.8, 29.4, 29.3, 29.2, 29.1, 28.6, 22.7, 14.1; IR (neat): ν=2923, 2853, 1949, 1490, 1463, 1090, 1013 cm⁻¹; MS (70 eV, EI) m/z (%): 264 (M⁺(³⁷Cl), 0.63), 262 (M⁺(³⁵Cl), 2.02), 129 (100).

### Example 39

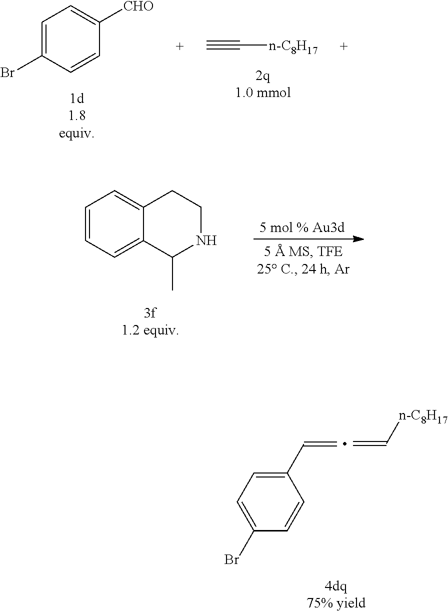

The operation is the same as Example 1. 5 Å molecular sieves (250.2 mg), Au3d (43.3 mg, 0.05 mmol), aldehyde 1d (333.5 mg, 1.8 mmol), 1-methyl-1,2,3,4-tetrahydroisoquinoline 3f (176.6 mg, 1.2 mmol) and terminal alkyne 2q (138.1 mg, 1.0 mmol) were reacted in 2,2,2-trifluoroethanol (1 mL) to afford an allene product 4dq (230.9 mg, 75% yield) (purified by flash column chromatography (eluent:petroleum ether)): oily liquid; ¹H NMR (400 MHz, CDCl₃): δ=7.40 (d, J=8.4 Hz, 2H, Ar—H), 7.14 (d, J=8.4 Hz, 2H, Ar—H), 6.06 (dt, J₁=6.3 Hz, J₂=3.1 Hz, 1H, ═CH), 5.56 (q, J=6.5 Hz, 1H, ═CH), 2.12 (qd, J₁=7.1 Hz, J₂=2.9 Hz, 2H, CH₂), 1.53-1.40 (m, 2H, CH₂), 1.40-1.14 (m, 10H, CH₂×5), 0.88 (t, J=6.8 Hz, 3H, CH₃); ¹³C NMR (100 MHz, CDCl₃): δ=205.2, 134.2, 131.5, 128.0, 120.1, 95.5, 93.8, 31.8, 29.4, 29.3, 29.2, 29.1, 28.6, 22.7, 14.1; IR (neat): ν=2922, 2852, 1949, 1486, 1462, 1069, 1009 cm⁻¹; MS (70 eV, EI) m/z (%): 308 (M⁺(⁸¹Br), 3.73), 306 (M⁺(⁷⁹Br), 4.13), 210 (100).

### Example 40

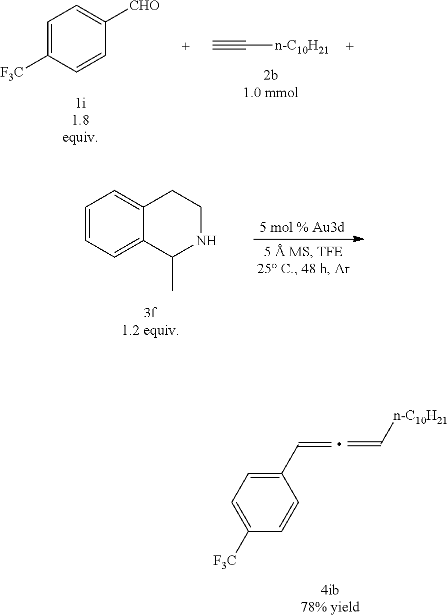

The operation is the same as Example 1. 5 Å molecular sieves (250.6 mg), Au3d (43.4 mg, 0.05 mmol), aldehyde 1i (313.2 mg, 1.8 mmol), 1-methyl-1,2,3,4-tetrahydroisoquinoline 3f (176.3 mg, 1.2 mmol) and terminal alkyne 2b (166.3 mg, 1.0 mmol) were reacted in 2,2,2-trifluoroethanol (1 mL) to afford an allene product 4ib (253.8 mg, 78% yield) (purified by flash column chromatography (eluent:petroleum ether)): oily liquid; ¹H NMR (400 MHz, CDCl₃): δ=7.53 (d, J=8.0 Hz, 2H, Ar—H), 7.37 (d, J=8.0 Hz, 2H, Ar—H), 6.14 (dt, J₁=6.1 Hz, J₂=3.0 Hz, 1H, ═CH), 5.63 (q, J=6.7 Hz, 1H, ═CH), 2.14 (qd, J₁=7.1 Hz, J₂=3.1 Hz, 2H, CH₂), 1.53-1.41 (m, 2H, CH₂), 1.41-1.12 (m, 14H, CH₂×7), 0.88 (t, J=6.8 Hz, 3H, CH₃); ¹³C NMR (100 MHz, CDCl₃): δ=206.2, 139.2, 128.6 (q, J=32.2 Hz), 126.6, 125.4 (q, J=3.8 Hz), 124.4 (q, J=269.6 Hz), 95.7, 93.8, 32.0, 29.71, 29.67, 29.5, 29.4, 29.2, 29.1, 28.5, 22.7, 14.1; ¹⁹F NMR (376 MHz, CDCl₃) δ=−62.9; IR (neat): ν=2924, 2854, 1949, 1616, 1322, 1163, 1123, 1065 cm⁻¹; MS (70 eV, EI) m/z (%): 324 (M⁺, 2.97), 198 (100).

### Example 41

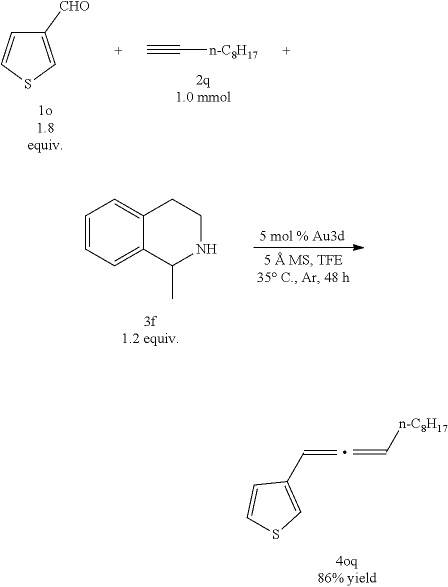

The operation is the same as Example 1. 5 Å molecular sieves (250.3 mg), Au3d (43.4 mg, 0.05 mmol), aldehyde to (202.1 mg, 1.8 mmol), 1-methyl-1,2,3,4-tetrahydroisoquinoline 3f (176.8 mg, 1.2 mmol) and terminal alkyne 2q (138.2 mg, 1.0 mmol) were reacted in 2,2,2-trifluoroethanol (1 mL) to afford an allene product 4oq (201.5 mg, 86% yield) (purified by flash column chromatography (eluent:Petroleum ether)): Oily liquid; ¹H NMR (400 MHz, CDCl₃): δ=7.27-7.21 (m, 1H, Ar—H), 7.06 (d, J=5.2 Hz, 1H, Ar—H), 7.04-6.99 (m, 1H, Ar—H), 6.18 (dt, J₁=6.3 Hz, J₂=3.0 Hz, 1H, ═CH), 5.48 (q, J=6.7 Hz, 1H, ═CH), 2.10 (qd, J₁=7.0 Hz, J₂=3.0 Hz, 2H, CH₂), 1.52-1.42 (m, 2H, CH₂), 1.40-1.12 (m, 10H, CH₂×5), 0.88 (t, J=6.6 Hz, 3H, CH₃); ¹³C NMR (100 MHz, CDCl₃): δ=205.4, 136.5, 126.2, 125.6, 120.0, 94.2, 89.1, 31.8, 29.4, 29.3, 29.2, 29.1, 28.8, 22.7, 14.1; IR (neat): ν=2922, 2852, 1950, 1461, 1378, 1233 cm⁻¹; MS (70 eV, EI) m/z (%): 234 (M⁺, 1.95), 135 (100).

### Example 42

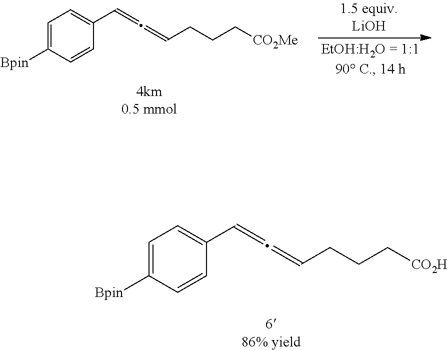

Lithium hydroxide (18.0 mg, 0.75 mmol), allene 4 km (171.3 mg, 0.5 mmol), ethanol (2.5 mL) and water (2.5 mL) were added in sequence to a reaction flask. After the reaction bottle was put into an oil bath at 90° C. to react for 14 hours, water (2 mL) and aqueous hydrochloric acid (1 M, 2 mL) were added in sequence to the reaction bottle. The mixture was extracted with ether (4 mL×5). The organic phases were combined, dried over anhydrous sodium sulfate, filtered, and concentrated to afford a product 6′ (151.9 mg, 93% purity, 86% yield): oily liquid; ¹H NMR (400 MHz, CDCl₃): δ=12.00-8.60 (br, 1H, CO₂H), 7.74 (d, J=8.0 Hz, 2H, Ar—H), 7.27 (d, J=7.6 Hz, 2H, Ar—H), 6.17 (dt, J₁=5.9 Hz, J₂=2.9 Hz, 1H, ═CH), 5.57 (q, J=6.5 Hz, 1H, ═CH), 2.43 (t, J=7.4 Hz, 2H, CH₂), 2.20 (qd, J₁=7.1 Hz, J₂=3.0 Hz, 2H, CH₂), 1.90-1.75 (m, 2H, CH₂), 1.34 (s, 12H, CH₃×4); ¹³C NMR (100 MHz, CDCl₃): δ=205.8, 179.8, 137.7, 135.0, 125.9, 95.3, 94.0, 83.7, 33.3, 27.8, 24.78, 24.75, 23.8; IR (neat): ν=2976, 1944, 1701, 1607, 1355, 1321, 1245, 1168, 1140, 1088 cm⁻¹; MS (FI) m/z (%): 328 (M⁺(¹¹B)); HRMS Calcd. for C₁₉H₂₅O₄¹⁰B (M⁺): 327.1877, found 327.1882.

### Example 43

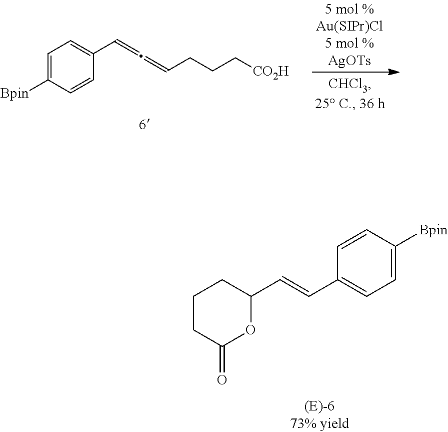

Au(SIPr)Cl (6.2 mg, 0.01 mmol), AgOTs (2.9 mg, 0.01 mmol) and chloroform (2 mL) were added in sequence to a reaction flask. After stirring at room temperature for 15 minutes, 6′ (70.1 mg, 93% purity, 0.2 mmol) and chloroform (1 mL) were added in sequence. After stirring at room temperature for 36 hours, the reaction solution was filtered through a short silica gel column, and the filter cake was washed with ethyl acetate (30 mL), concentrated, purified by flash column chromatography (eluent:petroleum ether/ethyl acetate=4/1) to afford a product (E)-6 (47.9 mg, 73% yield): oily liquid; ¹H NMR (400 MHz, CDCl₃): δ=7.77 (d, J=8.0 Hz, 2H, Ar—H), 7.39 (d, J=8.0 Hz, 2H, Ar—H), 6.68 (d, J=16.0 Hz, 1H, ═CH), 6.27 (dd, J₁=15.8 Hz, J₂=6.2 Hz, 1H, ═CH), 5.09-4.91 (m, 1H, OCH), 2.73-2.58 (m, 1H, one proton of CH₂), 2.58-2.46 (m, 1H, one proton of CH₂), 2.14-2.03 (m, 1H, one proton of CH₂), 2.03-1.84 (m, 2H, one proton of CH₂×2), 1.83-1.67 (m, 1H, one proton of CH₂), 1.34 (s, 12H, CH₃×4); ¹³C NMR (150 MHz, CDCl₃): δ=171.0, 138.6, 135.1, 132.0, 128.0, 125.9, 83.8, 80.2, 29.6, 28.4, 24.824, 24.816, 18.3; IR (neat): ν=2977, 1731, 1607, 1357, 1233, 1142, 1087, 1033 cm⁻¹; MS (FI) m/z (%): 328 (M⁺(¹¹B); HRMS Calcd. for C₁₉H₂₅O₄¹⁰B (M⁺): 327.1877, found 327.1879.

### Example 44

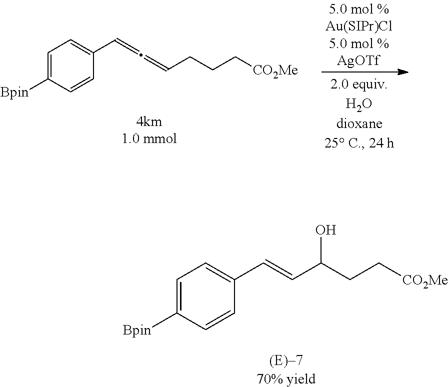

Au(SIPr)Cl (31.3 mg, 0.05 mmol), AgOTf (12.8 mg, 0.05 mmol) and dioxane (1.25 mL) were added in sequence to a reaction flask. After stirring at room temperature for 15 minutes, 4 km (342.0 mg, 1.0 mmol) and water (36.0 mg, 2.0 mmol) were added in sequence. After stirring at room temperature for 24 hours, the reaction solution was filtered through a short silica gel column, and the filter cake was washed with diethyl ether (30 mL), concentrated, and purified by flash column chromatography (eluent:petroleum ether/ethyl acetate=4/1) to afford a product (E)-7 (252.9 mg, 70% yield): oily liquid; ¹H NMR (400 MHz, CDCl₃): δ=7.76 (d, J=7.6 Hz, 2H, Ar—H), 7.37 (d, J=8.0 Hz, 2H, Ar—H), 6.59 (d, J=16.0 Hz, 1H, ═CH), 6.28 (dd, J₁=15.6 Hz, J₂=6.4 Hz, 1H, ═CH), 4.36-4.24 (m, 1H, CH), 3.67 (s, 3H, OCH₃), 2.38 (t, J=7.2 Hz, 2H, CH₂), 1.88-1.59 (m, 5H, CH₂×2 and OH), 1.34 (s, 12H, CH₃×4); ¹³C NMR (100 MHz, CDCl₃): δ=174.0, 139.3, 135.0, 133.1, 130.3, 125.7, 83.7, 72.4, 51.5, 36.5, 33.7, 24.8, 20.8; IR (neat): ν=3466, 2978, 1732, 1608, 1357, 1142, 1087 cm⁻¹; MS (FI) m/z (%): 360 (M⁺(¹¹B); HRMS Calcd. for C₂₀H₂₉O₅¹⁰B (M⁺): 359.2139, found 359.2148.

### Example 45

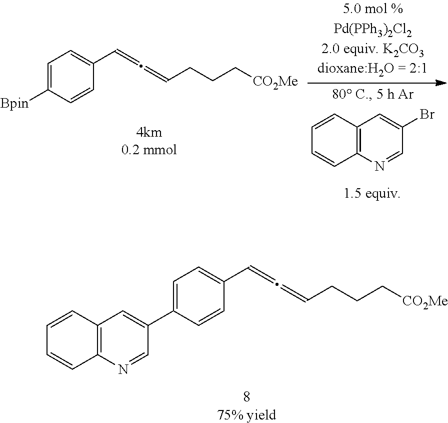

Under an argon atmosphere, Pd(PPh₃)₂Cl₂ (7.0 mg. 0.01 mmol), potassium carbonate (55.6 mg, 0.4 mmol), 4 km (68.4 mg, 0.2 mmol), 3-Bromoquinoline (62.7 mg, 0.3 mmol), freshly distilled dioxane (1 mL) and water (0.5 mL) were added in sequence to a dry reaction flask. The reaction flask was put into an oil bath at 80° C. for 5 hours. The reaction solution was filtered with a short silica gel column, and the filter cake was washed with ether, concentrated, and purified by flash column chromatography (eluent:petroleum ether/ethyl acetate=3/1) to afford a product 8 (54.3 mg, 95% purity, 75% yield): oily liquid; ¹H NMR (400 MHz, CDCl₃): δ=9.19 (d, J=2.4 Hz, 1H, Ar—H), 8.30 (d, J=2.0 Hz, 1H, Ar—H), 8.13 (d, J=8.4 Hz, 1H, Ar—H), 7.88 (d, J=8.0 Hz, 1H, Ar—H), 7.76-7.69 (m, 1H, Ar—H), 7.67 (d, J=8.4 Hz, 2H, Ar—H), 7.62-7.54 (m, 1H, Ar—H), 7.44 (d, J=8.4 Hz, 2H, Ar—H), 6.23 (dt, J₁=6.1 Hz, J₂=3.0 Hz, 1H, ═CH), 5.63 (q, J=6.5 Hz, 1H, ═CH), 3.68 (s, 3H, OCH₃), 2.42 (t, J=7.4 Hz, 2H, CH₂), 2.29-2.13 (m, 2H, CH₂), 1.92-1.80 (m, 2H, CH₂); ¹³C NMR (100 MHz, CDCl₃): δ=205.6, 173.8, 149.7, 147.2, 136.1, 134.8, 133.4, 132.7, 129.25, 129.16, 128.0, 127.9, 127.5, 127.3, 126.9, 94.6, 94.3, 51.5, 33.3, 28.0, 24.2; IR (neat): ν=2948, 1946, 1731, 1515, 1492, 1435, 1340, 1195, 1149 cm⁻¹; MS (ESI) m z (%): 344 (M+H⁺); HRMS Calcd. for C₂₃H₂₂NO₂ (M+H⁺): 344.1645, found 344.1644.

### Example 46

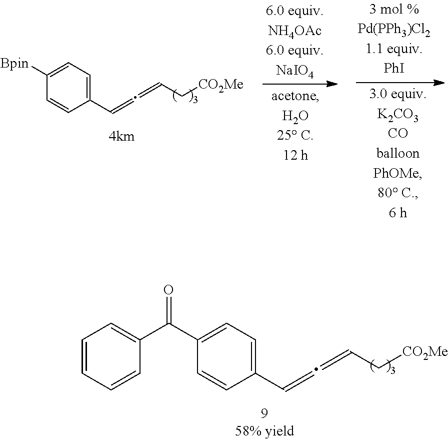

4 km (68.8 mg, 0.2 mmol), ammonium acetate (92.6 mg, 1.2 mmol), sodium periodate (257.1 mg, 1.2 mmol), acetone (2.4 mL) and distilled water (1.2 mL) were added in sequence to a reaction flask. After reacting at room temperature for 12 hours, the reaction solution was filtered with a short silica gel column, and the filter cake was washed with acetone (15 mL), concentrated, extracted with diethyl ether (4 mL×3), and the organic phases were combined, dried over anhydrous sodium sulfate, filtered, concentrated, purified by flash column chromatography (eluent dichloromethane/methanol=80/1) to afford the corresponding boronic acid (47.9 mg).

Pd(PPh₃)₂Cl₂ (3.9 mg, 5.56×10⁻³ mmol), potassium carbonate (76.5 mg, 0.554 mmol) and the above prepared boronic acid (47.9 mg, 0.184 mmol) were added in sequence to a dry reaction flask. After the reaction tube was plugged with a rubber stopper, inserted with a carbon monoxide balloon, connected to a vacuum pump, and replaced with the carbon monoxide three times under a carbon monoxide atmosphere. Then 1.1 mL of iodobenzene (41.4 mg, 0.203 mmol) in anisole solution was added. The reaction flask was placed in an oil bath at 80° C. and stirred for 6 hours. The reaction solution was filtered through a short silica gel column, and the filter cake was washed with diethyl ether (15 mL), concentrated, and purified by flash column chromatography (eluent:petroleum ether/ethyl acetate=20/1) to afford a product 9 (37.3 mg, 58% yield): oily liquid; ¹H NMR (400 MHz, CDCl₃): δ=7.78 (t, J=8.4 Hz, 4H, Ar—H), 7.62-7.53 (m, 1H, Ar—H), 7.48 (t, J=7.6 Hz, 2H, Ar—H), 7.38 (d, J=8.4 Hz, 2H, Ar—H), 6.22 (dt, J₁=6.3 Hz, J₂=3.1 Hz, 1H, ═CH), 5.64 (q, J=6.5 Hz, 1H, ═CH), 3.67 (s, 3H, OCH₃), 2.41 (t, J₁=7.4 Hz, 2H, CH₂), 2.21 (qd, J₁=7.1 Hz, J₂=2.9 Hz, 2H, CH₂), 1.91-1.78 (m, 2H, CH₂); ¹³C NMR (100 MHz, CDCl₃): δ=206.5, 196.2, 173.8, 139.5, 137.8, 135.7, 132.2, 130.6, 129.9, 128.2, 126.3, 94.7, 94.6, 51.6, 33.3, 27.8, 24.1; IR (neat): ν=2949, 1945, 1732, 1652, 1599, 1435, 1306, 1275, 1174, 1148 cm⁻¹; MS (ESI) m/z (%): 321 (M+H⁺); HRMS Calcd. for C₂₁H₂₁O₃ (M+H⁺): 321.1485, found 321.1484.

### Example 47

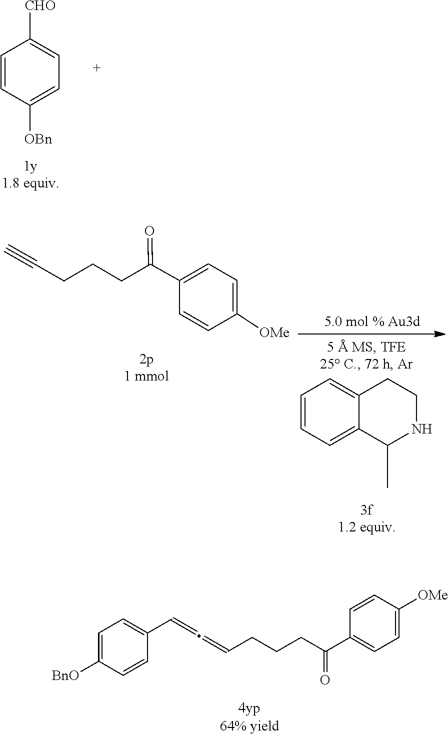

The operation is the same as Example 1. 5 Å molecular sieves (250.4 mg), Au3d (43.5 mg, 0.05 mmol), aldehyde 1y (382.3 mg, 1.8 mmol), 1-methyl-1,2,3,4-tetrahydroisoquinoline 3f (176.5 mg, 1.2 mmol) and terminal alkyne 2p (202.1 mg, 1.0 mmol) were reacted in 2,2,2-trifluoroethanol (1 mL) to afford an allene product 4yp (255.2 mg, 64% yield) (purified by flash column chromatography (eluent:petroleum ether/ethyl acetate=40/1)): white solid; m.p. 71.0-72.2° C. (recrystallized from ether); ¹H NMR (400 MHz, CDCl₃): δ=7.89 (d, J=8.8 Hz, 2H, Ar—H), 7.45-7.34 (m, 4H, Ar—H), 7.34-7.27 (m, 1H, Ar—H), 7.20 (d, J=8.4 Hz, 2H, Ar—H), 6.89 (d, J=8.8 Hz, 4H, Ar—H), 6.15-6.06 (m, 1H, ═CH), 5.56 (q, J=6.5 Hz, 1H, ═CH), 5.03 (s, 2H, CH₂), 3.85 (s, 3H, OCH₃), 2.97 (t, J=7.4 Hz, 2H, CH₂), 2.28-2.16 (m, 2H, CH₂), 1.92 (quintet, J=7.1 Hz, 2H, CH₂); ¹³C NMR (100 MHz, CDCl₃): δ=204.6, 198.5, 163.2, 157.7, 136.8, 130.2, 130.0, 128.4, 127.8, 127.6, 127.31, 127.28, 115.0, 113.5, 94.30, 94.25, 69.9, 55.3, 37.3, 28.4, 23.5; IR (neat): ν=2897, 2840, 1943, 1670, 1602, 1577, 1509, 1231, 1167 cm⁻¹; MS (70 eV, EI) m/z (%): 398 (M⁺, 3.73), 91 (100).

Those skilled in the art will understand that within the protection scope of the present invention, it is feasible to modify, add and replace the above-mentioned embodiments, and none of them exceeds the protection scope of the present invention.

1. A method for preparing 1,3-disubstituted allene compound based on a gold carbene catalytic system at room temperature, wherein, terminal alkynes (2) with different substituents, aldehydes (1) and amines (3) in an organic solvent under the action of a gold carbene catalyst and a molecular sieve to generate 1,3-disubstituted allene compound at room temperature, the reaction process is shown in the following reaction formula (a): 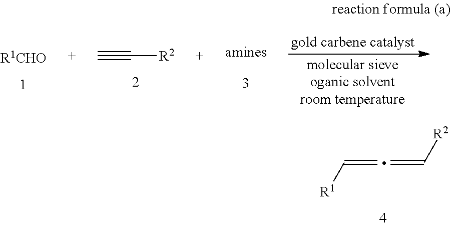
wherein, R¹ is C1-C10 alkyl group, C1-C10 alkyl group with functional groups, phenyl, aryl and heterocyclic groups; R² is C1-C10 alkyl group, C1-C10 alkyl group with functional groups, phenyl, aryl and heterocyclic groups; the functional groups in R¹ and R² are selected from carbon-carbon double bond, halogen atom, hydroxyl, silyl ether, carbonyl, nitrile, ester and amido groups; the said aryl groups are phenyl and polyphenyl cyclosubstituented groups with electron-donating or electron-withdrawing substituents in the ortho, meta and para positions; the said electron-donating substituents comprises alkyl, methoxy, benzyloxy and boronate groups, and the said electron-withdrawing substituents comprises halogen, nitrile, ester, trifluoromethyl and nitro groups; the said heterocyclic groups are furyl, benzofuryl, thienyl, pyridyl, indolyl and indazolyl.

2. The method of claim 1, wherein, the method specifically comprises the following steps: under an argon atmosphere, molecular sieves, gold carbene catalysts and a certain volume of organic solvent are added in sequence to the dry reaction tube; the aldehydes (1), amines (3) and terminal alkynes (2) are then added in sequence with stirring, reaction is carried out for 24-72 hours at room temperature; after the reaction is complete, the reaction mixture was filtered through a short silica gel column and washed with a certain volume of diethyl ether; after the mixture was concentrated, it was subjected to flash column chromatography to obtain 1,3-disubstituted allene compound;
wherein, the organic solvent of a certain volume refers to the amount of the terminal alkynes (2) shown in the formula (a) as a benchmark, the amount of the organic solvent is 0.5-5 mL/mmol; wherein, the room temperature means 10-40° C.; the said certain volume of diethyl ether refers to the amount of terminal alkynes (2) shown in the formula (a) as a benchmark, the amount of the said diethyl ether is 10-100 mL/mol.

3. The method of claim 1, wherein, the amines (3) is selected from morpholine(3a), piperidine(3b), pyrrolidine(3c), 1,2,3,4-tetrahydroquinoline(3d), 1,2,3,4-tetrahydroisoquinoline(3e), 1-methyl-1,2,3,4-tetrahydroisoquinoline(3f), diethylamine(3g), diallylamine(3h), dicyclohexylamine(3i) and diisopropylamine(3j).

4. The method of claim 1, wherein, the gold carbene catalyst is selected from one or more of the following structures Au1-Au3, wherein, R is C1-C30 alkyl group, C1-C30 alkyl group with functional groups, phenyl, aryl, and heterocyclic; the functional group is selected from carbon-carbon double bond, carbon-carbon triple bond, halogen atom, hydroxyl, carboxyl, amino, silyl ether, carbonyl, nitrile, ester and amido groups; the said aryl refers to phenyl and polyphenyl cyclosubstituents with electron-donating or electron-withdrawing substituents in the ortho, meta, and para positions; the electron-donating substituents include alkyl, alkoxy and boronate groups, and the electron-withdrawing substituents include halogen, nitrile, ester, trifluoromethyl and nitro groups; among them, X is a counter anion, including halogen anion, hydroxide anion, bis(trifluoromethanesulfonyl)imide anion, methanesulfonate anion, trifluoromethanesulfonate anion, p-methylbenzenesulfonate anion, perchlorate anion, tetrafluoroborate anion, hexafluorophosphate anion and hexafluoroantimonate anion;

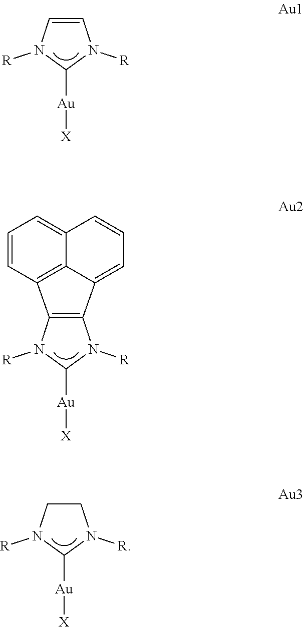

5. The method of claim 1, wherein, the molecular sieve is from 3 Å molecular sieve, 4 Å molecular sieve and 5 Å molecular sieve.

6. The method of claim 1, wherein, the organic solvent is selected from one or more of 2,2,2-trifluoroethanol, 2,2,3,3-tetrafluoropropanol, 2,2,3,3,3-pentafluoropropanol and 1,1,1,3,3,3-hexafluoroisopropanol.

7. The method of claim 1, wherein, the mol ratio of the terminal alkynes (2), aldehydes (1), amines (3) and gold carbene catalyst is 1.0:(1.0-1.8):(1.0-1.4):(0.01-0.1); and/or, the amount of the molecular sieve is 100-500 mg/mmol, based on the amount of the terminal alkynes (2); and/or, the amount of the organic solvent is 0.5-5 mL/mmol, based on the amount of the terminal alkynes (2).

8. The method of claim 1, wherein, the room temperature refers to 10-60° C.; the reaction time is 24-72 hours.

9. A class of 1,3-disubstituted allene compound, wherein, its structure is shown in the following formula 4: 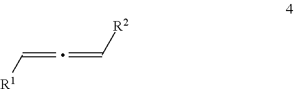
wherein, R¹ is C1-C10 alkyl group, C1-C10 alkyl group with functional groups, phenyl, aryl and heterocyclic groups; R² is C1-C10 alkyl group, C1-C10 alkyl group with functional groups, phenyl, aryl and heterocyclic groups; the functional groups in R¹ and R² are selected from carbon-carbon double bond, halogen atom, hydroxyl, silyl ether, carbonyl, nitrile, ester and amido groups; the aryl groups are phenyl and polyphenyl cyclosubstituented groups with electron-donating or electron-withdrawing substituents in the ortho, meta and para positions; the said electron-donating substituents comprises alkyl, methoxy, benzyloxy and boronate groups, and the said electron-withdrawing substituents comprises halogen, nitrile, ester, trifluoromethyl and nitro groups; the said heterocyclic groups are furyl, benzofuryl, thienyl, pyridyl, indolyl and indazolyl.

10. The application of the 1,3-disubstituted allene compound according to claim 9 in the preparation of δ-caprolactone, trans-allyl alcohol, other allene derived compounds, and natural product molecules.

11. The method of claim 2, wherein, the amines (3) is selected from morpholine(3a), piperidine(3b), pyrrolidine(3c), 1,2,3,4-tetrahydroquinoline(3d), 1,2,3,4-tetrahydroisoquinoline(3e), 1-methyl-1,2,3,4-tetrahydroisoquinoline(3f), diethylamine(3g), diallylamine(3h), dicyclohexylamine(3i) and diisopropylamine(3j).

12. The method of claim 2, wherein, the gold carbene catalyst is selected from one or more of the following structures Au1-Au3, wherein, R is C1-C30 alkyl group, C1-C30 alkyl group with functional groups, phenyl, aryl, and heterocyclic; the functional group is selected from carbon-carbon double bond, carbon-carbon triple bond, halogen atom, hydroxyl, carboxyl, amino, silyl ether, carbonyl, nitrile, ester and amido groups; the said aryl refers to phenyl and polyphenyl cyclosubstituents with electron-donating or electron-withdrawing substituents in the ortho, meta, and para positions; the electron-donating substituents include alkyl, alkoxy and boronate groups, and the electron-withdrawing substituents include halogen, nitrile, ester, trifluoromethyl and nitro groups; among them, X is a counter anion, including halogen anion, hydroxide anion, bis(trifluoromethanesulfonyl)imide anion, methanesulfonate anion, trifluoromethanesulfonate anion, p-methylbenzenesulfonate anion, perchlorate anion, tetrafluoroborate anion, hexafluorophosphate anion and hexafluoroantimonate anion;

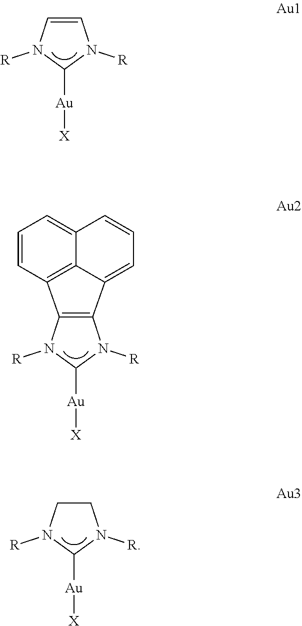

13. The method of claim 2, wherein, the molecular sieve is from 3 Å molecular sieve, 4 Å molecular sieve and 5 Å molecular sieve.

14. The method of claim 2, wherein, the organic solvent is selected from one or more of 2,2,2-trifluoroethanol, 2,2,3,3-tetrafluoropropanol, 2,2,3,3,3-pentafluoropropanol and 1,1,1,3,3,3-hexafluoroisopropanol.
